(19) United States
(12) Patent Application Publication
(10) Pub. No.: US 2025/0154519 A1
(43) Pub. Date: May 15, 2025

(54) REGULATION OF GENE EXPRESSION

(71) Applicants: THE NEW ZEALAND INSTITUTE FOR PLANT AND FOOD RESEARCH LIMITED, Auckland (NZ); WUHAN BOTANICAL GARDEN, CHINESE ACADEMY OF SCIENCES, Wuhan (CN)

(72) Inventors: Dawei LI, Wuhan (CN); Xiaoying LIU, Wuhan (CN); Caihong ZHONG, Wuhan (CN); Xiaodong XIE, Wuhan (CN); Sean Michael Winsley BULLEY, Auckland (NZ)

(73) Assignees: THE NEW ZEALAND INSTITUTE FOR PLANT AND FOOD RESEARCH LIMITED, Auckland (NZ); WUHAN BOTANICAL GARDEN, CHINESE ACADEMY OF SCIENCES, Wuhan (CN)

(21) Appl. No.: 18/838,193

(22) PCT Filed: Feb. 14, 2023
(86) PCT No.: PCT/IB2023/051306

§ 371 Date: Aug. 13, 2024

(30) Foreign Application Priority Data
Feb. 14, 2022 (AU) 2022900309

(51) Int. Cl. C12N 15/82

(52) U.S. Cl. CPC C12N 15/8243; C12N 15/8205

(57) ABSTRACT

The invention provides a method of producing a host cell, plant cell or plant with increased L-ascorbic acid (AsA) content or increased GDP-L-galactose phosphorylase (GGP) translation, production and/or activity, the method comprising transformation of the host cell or plant cell with a polynucleotide encoding a polypeptide that regulates GDP-L-galactose phosphorylase (GGP). The invention also provides host cells, plant cells and plants, genetically modified to contain and/or express the polynucleotides.

### FIELD OF THE INVENTION

The present invention relates to polynucleotides, genetic constructs and methods for producing plants with altered GDP-L-galactose phosphorylase 3 (GGP) translation, production and/or activity; and/or altered L-ascorbic acid (AsA) content; and plants and plant cells produced from said methods.

### BACKGROUND TO THE INVENTION

L-ascorbic acid (AsA) is the most abundant soluble antioxidant in plants and is also an essential nutrient for humans and a few other animals. AsA contributes significantly to the overall intake of “free radical scavengers” or “anti-oxidative metabolites” in the human diet. Convincing evidence now shows that such metabolites either singly or in combination, benefit health and well-being, acting as anti-cancer forming agents and protecting against coronary heart disease. Eating sufficient amounts of fruits and vegetables to consistently maintain optimum AsA concentrations is still a challenge for people in both developed and developing countries.

Almost the entire dietary AsA intake in humans is derived from plant products. However, the AsA content of plant tissues is remarkably variable. Whilst leaf AsA content is generally high and relatively uniform in herbaceous and woody plants, a huge and unexplained variability in AsA content is found in non-green edible plant tissues.

Understanding how AsA biosynthesis is regulated may provide tools to manipulate biosynthesis in plants. Understanding the regulation of gene expression, and the factors/elements controlling such expression also provide valuable tools for genetic manipulation. The enzyme GDP-L-galactose phosphorylase (GGP) is known to play a key role in AsA biosynthesis. The regulation of GGP expression and activity is yet to be fully resolved.

It would be desirable to increase the AsA content of plants.

It is an object of the invention to provide new, improved and/or alternative tools for manipulating the expression and content of AsA in plants, and/or to at least provide the public with a useful choice.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### SUMMARY OF THE INVENTION

In a first aspect the invention relates to a method of producing a plant cell or plant with increased L-ascorbic acid (AsA) content, the method comprising transforming a plant cell with a polynucleotide encoding a polypeptide with an amino acid sequence of any one of SEQ ID NOs: 1-102 and 348-356, or a variant of the polypeptide having at least about 70% sequence identity to a polypeptide with the amino acid sequence of any one of SEQ ID NOS: 1-102 and 348-356.

In a second aspect the invention relates to a method of producing a plant cell or plant having increased GDP-L-galactose phosphorylase 3 (GGP) translation, production and/or activity, the method comprising transforming a plant cell with a polynucleotide encoding a polypeptide with an amino acid sequence of any one of SEQ ID NOs: 1-102 and 348-356, or a variant of the polypeptide having at least about 70% sequence identity to a polypeptide with the amino acid sequence of any one of SEQ ID NOs: 1-102 and 348-356.

In a third aspect the invention relates to a method of producing a plant cell or plant with increased L-ascorbic acid (AsA) content, the method comprising transforming a plant cell with a polynucleotide comprising a nucleotide sequence of any one of SEQ ID NOs: 103-108, or a variant thereof having at least about 70% sequence identity to a nucleotide having the nucleotide sequence of any one of SEQ ID NOs: 103-108.

In a fourth aspect the invention relates to a method of producing a plant cell or plant having increased GDP-L-galactose phosphorylase 3 (GGP) translation, production and/or activity, the method comprising transforming a plant cell with a polynucleotide comprising a nucleotide sequence of any one of SEQ ID NOs: 103-108, or a variant thereof having at least about 70% sequence identity to a nucleotide having the nucleotide sequence of any one of SEQ ID NOs: 103-108.

In a fifth aspect the invention relates to a method of producing a plant cell or plant with increased L-ascorbic acid (AsA) content or increased GDP-L-galactose phosphorylase 3 (GGP) translation, production and/or activity, the method comprising upregulating in the plant cell, or expressing in the plant, a polypeptide with the amino acid sequence of any one of SEQ ID NO: 1-102 and 348-356, or a variant of the polypeptide having at least about 70% sequence identity to the amino acid sequence of any one of SEQ ID NO: 1-102 and 348-356.

In a sixth aspect the invention relates to a method of producing a plant cell or plant with increased L-ascorbic acid (AsA) content or increased GDP-L-galactose phosphorylase 3 (GGP) translation, production and/or activity, the method comprising upregulating in the plant cell, or expressing in the plant, a polynucleotide comprising a nucleotide sequence selected from any one of the sequences SEQ ID NO: 103-108 or a variant having at least about 70% sequence identity to the nucleotide sequence of any one of SEQ ID NO: 103-108.

In a seventh aspect, the invention relates to an isolated polynucleotide encoding a polypeptide with the amino acid sequence of any one of SEQ ID NOs: 1-4 and 102, or a variant of the polypeptide having at least about 70% sequence identity to the amino acid sequence of any one of SEQ ID NOs: 1-4 and 102, preferably at least about 75% sequence identity.

In an eighth aspect, the invention relates to an isolated polynucleotide encoding a polypeptide with an amino acid sequence of any one of SEQ ID NOs: 109-153 155-157, 159, 162-164, 166-174, 176, 178, 180-183, 185, 186, 188-190, 192-200, 202-204, and 206-209, or a variant of the polypeptide having at least about 70% sequence identity to the amino acid sequence of any one of SEQ ID NOs: 109-153 155-157, 159, 162-164, 166-174, 176, 178, 180-183, 185, 186, 188-190, 192-200, 202-204, and 206-209, preferably at least about 75% sequence identity.

In a ninth aspect the invention relates to a genetic construct comprising a polynucleotide encoding a polypeptide with an amino acid sequence of any one of SEQ ID NOs: 1-4 and 102, or a variant of the polypeptide having at least about 70% sequence identity to the amino acid sequence of any one of SEQ ID NOS: 1-4 and 102, preferably at least about 75% sequence identity.

In a tenth aspect the invention relates to a genetic construct comprising a polynucleotide encoding a polypeptide with an amino acid sequence of any one of SEQ ID NOs: 109-153 155-157, 159, 162-164, 166-174, 176, 178, 180-183, 185, 186, 188-190, 192-200, 202-204, and 206-209, or a variant of the polypeptide having at least about 70% sequence identity to the amino acid sequence of any one of SEQ ID NOs: 109-153 155-157, 159, 162-164, 166-174, 176, 178, 180-183, 185, 186, 188-190, 192-200, 202-204, and 206-209, preferably at least about 75% sequence identity.

In various embodiments the genetic construct may further comprise a second polynucleotide
a) encoding a polypeptide with an amino acid sequence of any one of SEQ ID NOS: 109-209, or a variant of the polypeptide having at least about 70% sequence identity to the amino acid sequence of any one of SEQ ID NOs: 109-209, preferably at least about 75% sequence identity; and/or
b) having a nucleotide sequence of SEQ ID NO: 210, or a variant of the nucleotide having at least about 70% sequence identity to the nucleotide sequence of SEQ ID NO: 210.

In an eleventh aspect the invention relates to a genetic construct comprising a polynucleotide comprising a nucleotide sequence of any one of SEQ ID NOs: 103-108, or a variant of the polypeptide having at least about 70% sequence identity to the nucleotide sequence of any one of SEQ ID NOs: 103-108, preferably at least about 75% sequence identity.

In a twelfth aspect the invention relates to a genetic construct comprising a polynucleotide comprising a nucleotide sequence of SEQ ID NO: 210, or a variant of the polypeptide having at least about 70% sequence identity to the nucleotide sequence of SEQ ID NO: 210.

In a thirteenth aspect the invention relates to a kit comprising a first genetic construct of the ninth or eleventh aspects described herein; and a second genetic construct of the tenth or twelfth aspects described herein.

In a fourteenth aspect the invention provides a host cell comprising one or more genetic constructs described herein.

In a fifteenth aspect the invention provides a host cell genetically modified to express a polynucleotide encoding a polypeptide with an amino acid sequence of any one of SEQ ID NOS: 1-102 and 348-356, or a variant of the polypeptide having at least about 70% sequence identity to the amino acid sequence of any one of SEQ ID NOs: 1-102 and 348-356.

In a sixteenth aspect the invention provides a host cell genetically modified to express a polynucleotide comprising a nucleotide sequence of any one of SEQ ID NOS: 103-108, or a variant thereof having at least about 70% sequence identity to the nucleotide sequence of any one of SEQ ID NOs: 103-108.

In a seventeenth aspect the invention provides a plant cell genetically modified to express a polynucleotide encoding a polypeptide with the amino acid sequence of any one of SEQ ID NO: 1-102 and 348-356 or a variant of the polypeptide having at least 70% sequence identity to a polypeptide with the amino acid sequence of any one of SEQ ID NO: 1-102 and 348-356.

In an eighteenth aspect the invention provides a plant cell genetically modified to express a polynucleotide comprising a nucleotide sequence selected from any one of SEQ ID NO: 103-108 or a variant thereof having at least 70% sequence identity to the nucleotide sequence of any one of SEQ ID NO: 103-108.

In a nineteenth aspect the invention provides a plant comprising a plant cell described herein.

In a twentieth aspect the invention provides a method for selecting a plant with altered GDP-L-galactose phosphorylase 3 (GGP) activity, the method comprising testing a plant for altered expression of a polynucleotide encoding a polypeptide with the amino acid sequence of any one of SEQ ID NO: 1-102 and 348-356 or a variant of the polypeptide having at least 70% sequence identity to a polypeptide with the amino acid sequence of any one of SEQ ID NO: 1-102 and 348-356.

In a further aspect the invention provides a method for selecting a plant with altered GDP-L-galactose phosphorylase 3 (GGP) activity, the method comprising testing a plant for altered expression of a polynucleotide comprising a nucleotide sequence selected from any one of the sequences SEQ ID NO: 103-108 or a variant thereof wherein the variant comprises a sequence that has at least 70% sequence identity to the nucleotide sequence of any one of SEQ ID NO: 103-108.

In a further aspect the invention provides a method for selecting a plant with altered L-ascorbic acid (AsA) content, the method comprising testing a plant for altered expression of a polynucleotide encoding a polypeptide with the amino acid sequence of any one of SEQ ID NO: 1-102 and 348-356 or a variant of the polypeptide having at least 70% sequence identity to a polypeptide with the amino acid sequence of any one of SEQ ID NO: 1-102 and 348-356.

In a further aspect the invention provides a method for selecting a plant with altered L-ascorbic acid (AsA) content, the method comprising testing a plant for altered expression of a polynucleotide comprising a nucleotide sequence selected from any one of the sequences SEQ ID NO: 103-108 or a variant thereof comprising a sequence that has at least 70% sequence identity to the nucleotide sequence of any one of SEQ ID NO: 103-108.

In a further aspect the invention provides a plant cell or plant produced by any method described herein.

In a further aspect the invention provides a plant cell or plant selected by any method described herein.

In a further aspect the invention provides a group or population of plants produced by any method described herein.

In a further aspect the invention provides a method of producing AsA, the method comprising extracting AsA from a host cell described herein.

In a further aspect the invention provides a method of producing AsA, the method comprising extracting AsA from a plant cell or plant described herein.

The following embodiments may relate to any or all of the above aspects.

In various embodiments the method may further comprise transforming, or co-transforming with the polynucleotide, a second polynucleotide encoding a polypeptide with an amino acid sequence of any one of SEQ ID NOs: 109-209, or a variant of the polypeptide having at least about 70% sequence identity to the amino acid sequence of any one of SEQ ID NOs: 109-209.

In various embodiments the method may further comprise transforming, or co-transforming with the polynucleotide, a second polynucleotide with a nucleotide sequence of SEQ ID NO: 210, or a variant thereof having at least about 70% sequence identity to the nucleotide sequence of SEQ ID NO: 210. In various embodiments the variant has at least about 75, 80, 85, 90, 95, 96, 97, 98, 99 or 100% sequence identity to the nucleotide sequence of SEQ ID NO: 210.

In various embodiments the host cell or plant cell may be genetically modified to express a second polynucleotide encoding a polypeptide with an amino acid sequence of any one of SEQ ID NOs: 109-209, or a variant of the polypeptide having at least about 70% sequence identity to the amino acid sequence of any one of SEQ ID NOs: 109-209.

In various embodiments the host cell or plant cell may be genetically modified to express a second polynucleotide with a nucleotide sequence of SEQ ID NO: 210, or a variant thereof having at least about 70% sequence identity to the nucleotide sequence of SEQ ID NO: 210.

In various embodiments the polynucleotide may encode a polypeptide with an amino acid sequence of, or may encode a variant of the polypeptide having at least about 70, 75, 80, 85, 90, 95, 96, 97, 98, 99 or 100% sequence identity to an amino acid sequence of
a) any one of SEQ ID Nos: 1-102 and 348-356;
b) any one of SEQ ID Nos: 1-50, 52-60, 62-83, 85-95, 97-102, 348-350, and 352-354;
c) any one of SEQ ID Nos: 1-49, 53-56, 58-60, 62, 64-66, 70-79, 82, 83, 85-87, 89-91, 93-95, 98, 102, 348-350, 352, and 353;
d) any one of SEQ ID Nos: 1-49, 53-56, 58, 60, 62, 64, 66, 70, 71, 74-76, 78, 79, 83, 85-87, 89-91, 93-95, 98, 102, 348-350, 352, and 353;
e) any one of SEQ ID Nos: 1-49, 53-56, 58, 60, 62, 64, 66, 70, 71, 74-76, 78, 79, 83, 85-87, 89-90, 93, 94, 98, 102, 348-350, 352, and 353;
f) any one of SEQ ID Nos: 1-5, 7-10, 12-19, 21-24, 28-31, 33, 35, 36, 38-45, 48, 49, 53, 55, 56, 58, 60, 62, 64, 66, 71, 74-76, 79, 83, 85, 87, 89, 90, 93, 94, 98, 102, 350, 352;
g) any one of SEQ ID Nos: 1-4, 38, 41-45, 48, 49, 51, 52, 56, 60, 63, 66, 79, 80, 85, 89, 90, 92, 96, 97, 100-102, and 348-356;
h) any one of SEQ ID Nos: 1-4, 49, 51, 66, 80, 85, 90, 92, 96, 102, and 348-356;
i) any one of SEQ ID Nos: 1-4 and 102;
j) any one of SEQ ID Nos: 1-4;
k) SEQ ID No: 1 or 102;
l) SEQ ID No: 1; or
m) SEQ ID No: 102.

In various embodiments the polynucleotide may have a nucleotide sequence of, or may encode a variant of the polynucleotide having at least about 70, 75, 80, 85, 90, 95, 96, 97, 98, 99 or 100% sequence identity to a nucleotide sequence of:
a) any one of SEQ ID NOs: 103-108,
b) SEQ ID No: 103 or 105, or
c) SEQ ID No: 103.

In various embodiments the polynucleotide, or the second polynucleotide, may encode a polypeptide with an amino acid sequence of, or may encode a variant of the polypeptide having at least about 70, 75, 80, 85, 90, 95, 96, 97, 98, 99 or 100% sequence identity to an amino acid sequence of
a) any one of SEQ ID Nos: 109-209;
b) any one of SEQ ID Nos: 109-174, 176-178, and 180-209;
c) any one of SEQ ID Nos: 109-159, 162-164, 166-174, 176, 178, 180-186, 188-190, 192-204, and 206-209;
d) any one of SEQ ID Nos: 109-153 155-157, 159, 162-164, 166-174, 176, 178, 180-186, 188-190, 192-204, and 206-209;
e) any one of SEQ ID Nos: 109-153 155-157, 159, 162-164, 166-174, 176, 178, 180-183, 185, 186, 188-190, 192-200, 202-204, and 206-209;
f) any one of SEQ ID Nos: 109-114, 125, 126, 142, 177, 179, 187, 191 and 205;
g) any one of SEQ ID Nos: 109-114 and 179;
h) any one of SEQ ID Nos: 109-114; or
i) SEQ ID NO: 109.

In various embodiments the polynucleotide, or the second polynucleotide, may have a nucleotide sequence of, or may encode a variant of the polynucleotide having at least about 70, 75, 80, 85, 90, 95, 96, 97, 98, 99 or 100% sequence identity to a nucleotide sequence of SEQ ID NO: 210.

In various embodiments the polypeptide or polypeptide variant may comprise one or more SANT/MYB domains. In various embodiments the polypeptide or polypeptide variant may comprise two SANT/MYB domains.

In various embodiments the upregulating may comprise genetic engineering.

In various embodiments the transformation comprises stable transformation. In various embodiments, the method comprises stably transforming the plant cell with the polynucleotide or variant thereof. In various embodiments the host cell, or plant cell, is stably transformed and/or stably genetically modified.

In various embodiments the method may comprise transforming a plant cell with a genetic construct described herein. In various embodiments the method may comprise stably transforming a plant cell with a genetic construct described herein.

In various embodiments the host cell or plant cell comprises an endogenous GGP gene, preferably a functional GGP gene. For example, in some embodiments the host cell or plant cell has been previously transformed with a functional GGP gene. In some embodiments the method comprises transforming the host cell or plant cell with a GGP gene.

In some embodiments, the GGP gene has a promoter that comprises one or more MYBS1 binding sites, preferably two or more MYBS1 binding sites. In some embodiments, the method comprises gene-editing the promoter of an endogenous GGP gene to provide a promoter comprising one or more MYBS1 binding sites. In some embodiments, the method comprises modifying an endogenous GGP promoter to provide a GGP promoter comprising one or more MYBS1 binding sites. In some embodiments, the method comprises transforming the plant cell or host cell with a GGP gene. In some embodiments, the method comprises transforming the plant cell or host cell with a GGP gene having a promoter that comprises one or more MYBS1 binding sites, preferably two or more MYBS1 binding sites.

In various embodiments the upregulating may comprise crossing with a plant that expresses a polypeptide comprising an amino acid sequence having at least 70, 75, 80, 85, 90, 95, 96, 97, 98, or 99% sequence identity to a polypeptide with the amino acid sequence of any one of SEQ ID NO: 1-102 and 348-356 and/or SEQ ID No: 109-209.

According to some embodiments, the upregulating comprises crossing with a plant which expresses a polynucleotide comprising a nucleotide sequence selected from any one of the sequences SEQ ID NO: 103 to 108 and 210.

In various embodiments the genetic construct may comprise one or more polynucleotides operably linked to a promoter. In various embodiments the promoter is at least one of:
i) a promoter that is not normally associated with the polynucleotide in nature,
ii) a promoter derived from a bacterium, a fungus, an insect, a mammal or a virus.
iii) a bacterial promoter,
iv) a fungal promoter,
v) an insect promoter,
vi) a mammalian promoter, and
vii) a virus promoter.

In various embodiments the promoter may be derived from a bacterium, a fungus, an insect, a mammal or a virus.

In various embodiments, the promoter may be a constitutive promoter, or a tissue-specific promoter. In various embodiments, the promoter results in expression in leaves and/or roots. Preferably, the promoter results in expression in leaves. More preferably, the promoter is a tissue-specific promoter that results in targeted expression in leaves and/or roots.

In various embodiments the one or more polynucleotides may be regulatable by a compound. In some embodiments the compound is L-ascorbic acid (AsA), or a related metabolite.

In various embodiments the host cell may be a plant cell.

The term “comprising” as used in this specification and claims means “consisting at least in part of”. When interpreting statements in this specification and claims which include the term “comprising”, other features besides the features prefaced by this term in each statement can also be present. Related terms such as “comprise” and “comprised” are to be interpreted in similar manner.

As used herein the term “and/or” means “and” or “or”, or both.

As used herein the term ‘(s)’ following a noun means the plural and/or singular form of that noun.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

Although the present invention is broadly as defined above, those persons skilled in the art will appreciate that the invention is not limited thereto and that the invention also includes embodiments of which the following description gives examples.

### BRIEF DESCRIPTION OF THE DRAWINGS

The invention will now be described by way of example only and with reference to the drawings in which:

FIG. 1 provides a line graph demonstrating AsA content in six species of Actinidia in the days after flowering (DAF).

FIG. 2 provides graphs demonstrating the expression of GGP (GGP1, GGP2 and GGP3) (lines) and AsA content (bars) in (A) A. rufa and (B) A. eriantha in the days after flowering (DAF). Data is shown as the mean+/−standard deviation (n=3).

FIG. 3 provides bar graphs demonstrating that over-expression or knock down expression of AceGGP3 alters AsA concentration in A. eriantha fruits. (A) shows the expression abundance of AceGGP3 and (B) shows the AsA content in A. eriantha fruit 7 days after infiltration; and (C) shows the expression abundance of AceGGP3 and (D) shows the AsA content in A. eriantha calli 6 days after infiltration with: EV (empty vector); OX-AceGGP3 (AceGGP3 overexpression) or TRV-AceGGP3 (AceGGP3-antisense expression). Data is shown as the mean+/−standard deviation (n=3). Significant differences were detected by t-test using GraphPad Prism 8 (*, p<0.05; **, p<0.01; ***, p<0.001).

FIG. 4 provides bar graphs showing (A) RT-qPCR analysis of AceGGP3 and (B) relative AsA content in 5 transgenic lines of A. eriantha with AceGGP3-overexpressed. WT: wild-type; OE-GGP3 #10, OE-GGP3 #14, OE-GGP3 #20, OE-GGP3 #21, OE-GGP3 #23 represent the five different transgenic lines, respectively. Data is shown as the mean+/−standard deviation (n=3). Significant differences were detected by t-test using GraphPad Prism 8 (*, p<0.05; **, p<0.01; ***, p<0.001).

FIG. 5 provides a bar graph showing the relative AsA content of A. eriantha calli in CRISPR/Cas9-induced AceGGP3 mutants. wt: wild-type; ggp3 #2, ggp3 #11, ggp3 #13 and ggp3 #15 represent four gene editing lines, respectively. Data is shown as the mean+/−standard deviation (n=3). Significant differences were detected by t-test using GraphPad Prism 8 (*, p<0.05; **, p<0.01; ***, p<0.001).

FIG. 6 provides bar and line graphs showing a correlation between (A) AsA content (bars) and expression of AceMYBS1 (lines), and (B) the expression of AceGGP3 and AceMYBS1 in different developmental stage of A. eriantha fruits (every 20 days). Error bars represent +/−SD (n=3).

FIG. 7A provides a bar graph showing the results of dual-luciferase assays in tobacco leaves showing that AceMYBS1 activates transcription of different length fragments of AceGGP3 promoters (P₂₆₆₀, P₂₀₈₈, P₁₆₀₆, P₁₁₀₆). The empty vector (EV) is a control. Error bars: +/−SD. Significant differences were detected by t-test (**, p<0.01; ns: no significance). FIG. 7B shows the position of two AceMYBS1 predicted binding targets on the AceGGP3 promoter and electrophoretic mobility shift assay (EMSA) showing the binding of AceMYBS1 to the AceGGP3 promoter. The unlabelled probes were used as competitors. 100× and 300× represent the rates of the competitor.

FIG. 8 provides bar graphs showing the results of RT-qPCR analysis of (A) AceMYBS1 and (B) AceGGP3 transcripts, and (C) AsA content of A. eriantha fruits (of B) 7 days after infiltration with transient expressed-AceMYBS1 vectors. EV: empty vector; OX-AceMYBS1: AceMYBS1-overexpression; TRV-AceMYBS1: AceMYBS1-antisense expression. Experiments were repeated three times and each experiment contained three to six kiwifruits per genotype. Error bars: +/−SD. Significant differences were detected by t-test (*, p<0.05; **, p<0.01; ***, p<0.001; ns: no significance).

FIG. 9 provides bar graphs showing the results of RT-qPCR analysis of (A) AceMYBS1 and (B) AceGGP3; and (C) AsA content in A. eriantha calli transfected with the following vectors: EV: empty vector; OX-AceMYBS1: AceMYBS1-overexpression; TRV-AceMYBS1: AceMYBS1-antisense expression; TRV-AceGGP3: AceGGP3-antisense expression; TRV-AceGGP3+OX-AceMYBS1: overexpressed AceMYBS1 in TRV-AceGGP3 background. All experiments were performed in three replicates. Error bars: +/−SD. Significant differences were detected by t-test (*, p<0.05; **, p<0.01; ***, p<0.001; ns: no significance).

FIG. 10 provides bar graphs showing the results of RT-qPCR analysis of (A) AceMYBS1 and (B) AceGGP3 in wild-type (WT) and six AceMYBS1-overexpression transgenic kiwifruit (A. eriantha) lines: OE-AceMYBS1 #1, OE-AceMYBS1 #2, OE-AceMYBS1 #3, OE-AceMYBS1 #4, OE-AceMYBS1 #5, OE-AceMYBS1 #7.

FIG. 11 provides bar graphs showing the results of (A) bimolecular luminescence complementation (BiLC) assay demonstrating that AceMYBS1 interacts with AceGBF3 in vivo and (B) an in vitro pull-down assay showing the interaction between AceMYBS1 and AceGBF3. In (A), Agrobacterium clones containing the respective recombinant plasmids were combined at 1:1 (v/v) and infiltrated into N. benthamiana leaves. Error bars show the mean+/−SD; Significant differences were detected by t-test: ***, p<0.001; ns: no significance. In (B), AceGBF3-GST protein was incubated with immobilized AceMYBS1-6×His or 6×His protein, and immuno-precipitated fractions were detected by Anti-GST antibody.

FIG. 12 provides bar graphs showing the results of (A) dual luciferase assays in tobacco leaves showing the transcription of AceGGP3 activated by AceMYBS1 and AceGBF3 individually or collectively; (B) AsA content and (C) RT-qPCR analysis of the expression level in transiently expressed A. eriantha fruits 7 days post-transformation; the expression level in transiently expressed A. eriantha calli of (D) AceGGP3 and (E) AsA content; and the expression level in transiently expressed tobacco leaves of (F) NbGGP and (G) AsA content. EV: empty vector; OX-AceGBF3: AceGBF3-overexpression; TRV-AceGBF3: AceGBF3-antisense expression. The experiments were repeated three times and each experiment contain three to six kiwifruits per genotype. All above error bars denoted standard deviation (+/−SD), and performed three technical repeats each experimental group. Significant differences were detected by t-test (*, p<0.05; ** p<0.01; ***, p<0.001; ns: no significance). Different letters above the bars indicated significant difference (p<0.05) as obtain by one-way ANOVA test.

FIG. 13 provides bar graphs showing (A) AceGGP3 expression level as determined by RT-qPCR analysis and (B) AsA content in AceMYBS1 and AceGBF3 transiently co-expressed A. eriantha fruits. EV: empty vector; TRV-AceMYBS1: AceMYBS1-antisense expression; OX-AceMYBS1: AceMYBS1-overexpression; TRV-AceMYBS1+TRV-AceGBF3: co-antisense expression AceMYBS1 and AceGBF3; OX-AceMYBS1+OX-AceGBF3: co-overexpression AceMYBS1 and AceGBF3; OX-AceMYBS1+TRV-AceGBF3: AceMYBS1-overexpression in the AceGBF3-antisense expression background; OX-AceGBF3+TRV-AceMYBS1: AceGBF3-overexpression in the AceMYBS1-antisense expression background. The experiments were repeated three times and each experiment contain three to six kiwifruits per genotype. Significant differences were detected by t-test (*, p<0.05; **, p<0.01; ***, p<0.001; ns: no significance).

FIG. 14 provides bar graphs showing (A) relative AsA content and (B) RT-qPCR analysis of AceGGP3 in wild type (WT) and transgenic A. eriantha calli overexpressing AceGBF3. All above error bars denoted standard deviation (+/−SD), and performed three technical repeats each experimental group. Significant differences were detected by t-test (*, p<0.05; **, p<0.01; ***, p<0.001; ns: no significance).

FIG. 15 provides bar graphs showing the mean leaf AsA content (in mg/100 g fresh weight) of (A) rice, (B) soybean, and (C) Arabidopsis thaliana plants stably transformed to express AceMYBS1. Individual transformed lines are shown as separate bars. Measurements were performed in triplicate, and error bars denote the standard deviation. In (A) and (B), asterisks denote significant difference from wild-type according to Student's T-test (p<0.05). In (C), different letters denote significantly different groupings according to Student's T-test (p<0.05). WT: wild-type control plants.

### DETAILED DESCRIPTION OF THE INVENTION

The present invention, in some embodiments thereof, relates to methods for producing host cells, including plant cells or plants, having increased L-ascorbic acid (AsA) and/or increased GGP translation, production and/or activity.

The present invention is based on the identification, through genetic and molecular characterisation described herein, of two bZIP transcription factors that are positive regulators of AsA biosynthesis by activating transcription of GGP. An MYBS1-like transcription factor in kiwifruit was shown to bind the promoter of GGP3 and when overexpressed in kiwifruit resulted in significantly increased GGP3 expression and AsA accumulation. Overexpression of a GBF3 bZIP transcription factor also increased AsA content in an additive manner with MYBS1.

Identification of transcription factors that contribute to the regulation of AsA biosynthesis in plants allows for marker aided selection to be developed to breed plants having higher AsA content, and for high levels of AsA to be produced via biotechnological means, such as biopharming in crop plants and metabolic engineering of host cells such as yeast.

Thus, according to one aspect of the invention there is provided a method of producing a plant cell or plant with increased L-ascorbic acid (AsA) content, and/or increased GDP-L-galactose phosphorylase 3 (GGP) translation, production and/or activity, the method comprising transforming a plant cell with a polynucleotide encoding a polypeptide with an amino acid sequence of any one of SEQ ID NOS: 1-102 and 348-356, or a variant of the polypeptide having at least about 70% sequence identity to a polypeptide with the amino acid sequence of any one of SEQ ID NOs: 1-102 and 348-356 (MYBS1-like protein).

In various embodiments the method may further comprise transforming, or co-transforming with the polynucleotide, a second polynucleotide encoding a polypeptide with an amino acid sequence of any one of SEQ ID NOs: 109-209, or a variant of the polypeptide having at least about 70% sequence identity to the amino acid sequence of any one of SEQ ID NOS: 109-209 (GBF3-like protein).

The applicants have identified a polynucleotide (SEQ ID No: 103) encoding an MYBS1-like transcription factor polypeptide (SEQ ID No:1) and a polynucleotide (SEQ ID No: 210) that encodes a GBF3-like transcription factor polypeptide (Seq ID No: 109) from Actinidia eriantha as described in the examples.

The applicants have shown that these polypeptides induce expression of GGP3 and increase AsA content in several kiwifruit species and a tobacco species.

The applicants have also identified
a) MYBS1-like polypeptide sequences from a number of species that have significant sequence conservation with SEQ ID No: 1 and are variants of each other (SEQ ID Nos: 2-102 and 348-356), and
b) GBF3-like polypeptide sequences from a number of species that have significant sequence conservation with SEQ ID No: 109 and are variants of each other (SEQ ID Nos: 110-209).

Genetic constructs, vectors and plants containing polynucleotide sequences encoding an MYBS1-like polypeptide (SEQ ID NOs: 1-102 and 348-356) or sequences encoding the polypeptide sequences (SEQ ID NO: 103-108) and/or polynucleotide sequences encoding a GBF3-like polypeptide (SEQ ID Nos: 109-209) or sequences encoding the polypeptide sequences (SEQ ID NO: 210) are disclosed herein.

In certain embodiments, there are provided plants and host cells comprising the genetic constructs and vectors disclosed herein. Preferably the plants and host cells are stably transformed with the genetic constructs and/or vectors.

In some embodiments, there are provided plants altered GGP translation, production and/or activity relative to suitable control plants, and plants altered in AsA content relative to suitable control plants. In some embodiments, there are provided plants with increased GGP translation, production and/or activity and increased AsA. Preferably the plants are stably transformed or modified.

In other embodiments there are provided methods for the production of such plants and methods of selection of such plants.

Suitable control plants include non-transformed plants of the same species or variety or plants transformed with control constructs.

### 1. Polynucleotides and Fragments

The term “polynucleotide(s)” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers and fragments.

A “fragment” of a polynucleotide sequence provided herein is a subsequence of contiguous nucleotides that is capable of specific hybridization to a target of interest, e.g., a sequence that is at least 15 nucleotides in length. Fragments as herein disclosed comprise 15 nucleotides, preferably at least 20 nucleotides, more preferably at least 30 nucleotides, more preferably at least 50 nucleotides, more preferably at least 50 nucleotides and most preferably at least 60 nucleotides of contiguous nucleotides of a polynucleotide as herein disclosed. A fragment of a polynucleotide sequence can be used in antisense, gene silencing, triple helix or ribozyme technology, or as a primer, a probe, included in a microarray, or used in polynucleotide-based selection methods as herein disclosed.

The term “primer” refers to a short polynucleotide, usually having a free 3′OH group, that is hybridized to a template and used for priming polymerization of a polynucleotide complementary to the template. Such a primer is preferably at least 5, more preferably at least 6, more preferably at least 7, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20 nucleotides in length.

The term “probe” refers to a short polynucleotide that is used to detect a polynucleotide sequence, that is complementary to the probe, in a hybridization-based assay. The probe may consist of a “fragment” of a polynucleotide as defined herein. Preferably such a probe is at least 5, more preferably at least 10, more preferably at least 20, more preferably at least 30, more preferably at least 40, more preferably at least 50, more preferably at least 100, more preferably at least 200, more preferably at least 300, more preferably at least 400 and most preferably at least 500 nucleotides in length.

### 2. Polypeptides and Fragments

The term “polypeptide”, as used herein, encompasses amino acid chains of any length but preferably at least 5 amino acids, including full-length proteins, in which amino acid residues are linked by covalent peptide bonds. Polypeptides as herein disclosed may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof.

A “fragment” of a polypeptide is a subsequence of the polypeptide that performs a function that is required for the biological activity and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof capable of performing the above enzymatic activity.

The term “isolated” as applied to the polynucleotide or polypeptide sequences disclosed herein is used to refer to sequences that are removed from their natural cellular environment. An isolated molecule may be obtained by any method or combination of methods including biochemical, recombinant, and synthetic techniques.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

The term “derived from” with respect to polynucleotides or polypeptides as disclosed herein being derived from a particular genera or species, means that the polynucleotide or polypeptide has the same sequence as a polynucleotide or polypeptide found naturally in that genera or species. The polynucleotide or polypeptide, derived from a particular genera or species, may therefore be produced synthetically or recombinantly.

### 3. Variants

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. Variants described herein can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered variants.

In certain embodiments, variants of the polynucleotides and polypeptides disclosed herein possess biological activities that are the same or similar to those of the polynucleotides or polypeptides disclosed herein. The term “variant” with reference to polynucleotides and polypeptides encompasses all forms of polynucleotides and polypeptides as defined herein.

### 4. Polynucleotide Variants

Variant polynucleotide sequences preferably exhibit at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequence as disclosed herein. Identity is found over a comparison window of at least 20 nucleotide positions, preferably at least 50 nucleotide positions, more preferably at least 100 nucleotide positions, more preferably at least 200 nucleotide positions, more preferably at least 300 nucleotide positions, more preferably at least 400 nucleotide positions, more preferably at least 500 nucleotide positions, and most preferably over the entire length of a polynucleotide disclosed herein.

Polynucleotide sequence identity can be determined in the following manner. The subject polynucleotide sequence is compared to a candidate polynucleotide sequence using BLASTN (from the BLAST suite of programs, version 2.2.5 [November 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq are utilized except that filtering of low complexity parts should be turned off.

The identity of polynucleotide sequences may be examined using the following unix command line parameters:
bl2seq-i nucleotideseq1-j nucleotideseq2-F F-p blastn

The parameter-F F turns off filtering of low complexity sections. The parameter-p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences online at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Another method for calculating polynucleotide % sequence identity is based on aligning sequences to be compared using Clustal X (Jeanmougin et al., 1998, Trends Biochem. Sci. 23, 403-5.)

Polynucleotide variants disclosed herein also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs described supra.

The similarity of polynucleotide sequences may be examined using the following unix command line parameters:
bl2seq-i nucleotideseq1-j nucleotideseq2-F F-p tblastx

The parameter-F F turns off filtering of low complexity sections. The parameter-p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10⁻¹⁰ more preferably less than 1×10⁻²⁰, more preferably less than 1×10⁻³⁰, more preferably less than 1×10⁻⁴⁰, more preferably less than 1×10⁻⁵⁰, more preferably less than 1×10⁻⁶⁰, more preferably less than 1×10⁻⁷⁰, more preferably less than 1×10⁻⁸⁰, more preferably less than 1×10⁻⁹⁰ and most preferably less than 1×10⁻¹⁰⁰ when compared with any one of the specifically identified sequences.

Alternatively, variant polynucleotides as disclosed herein hybridize to the specified polynucleotide sequences, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0.41% (G+C−log (Na+). (Sambrook et al., Eds, 1987, Molecular Cloning, A Laboratory Manual, 2nd Ed. Cold Spring Harbor Press; Bolton and Mccarthy, 1962, PNAS 84:1390). Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length° C.)

With respect to the DNA mimics known as peptide nucleic acids (PNAs) (Nielsen et al., Science. 1991 Dec. 6; 254 (5037): 1497-500) Tm values are higher than those for DNA-DNA or DNA-RNA hybrids, and can be calculated using the formula described in Giesen et al., Nucleic Acids Res. 1998 Nov. 1; 26 (21): 5004-6. Exemplary stringent hybridization conditions for a DNA-PNA hybrid having a length less than 100 bases are 5 to 10° C. below the Tm.

Variant polynucleotides as disclosed herein also encompass polynucleotides that differ from the sequences as herein disclosed but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon usage in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available bl2seq program from the BLAST suite of programs (version 2.2.5 [November 2002]) from NCBI (ftp://ftp.ncbi.nih.gov/blast/) via the tblastx algorithm as previously described.

The function of the polypeptide encoded by a variant polynucleotide disclosed herein as modifier of GGP translation, production and/or activity may be assessed for example by expressing such a sequence in a host cell and testing its activity as described herein in the Examples. Function of a variant may also be tested for its ability to alter AsA content in plants, also as described in the Examples section herein.

### 5. Polypeptide Variants

The term “variant” with reference to polypeptides encompasses naturally occurring, recombinantly and synthetically produced polypeptides. Variant polypeptide sequences preferably exhibit at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequences of the present invention. Identity is found over a comparison window of at least 20 amino acid positions, preferably at least 50 amino acid positions, more preferably at least 100 amino acid positions, and most preferably over the entire length of a polypeptide as herein disclosed.

Polypeptide sequence identity can be determined in the following manner. The subject polypeptide sequence is compared to a candidate polypeptide sequence using BLASTP (from the BLAST suite of programs, version 2.2.5 [November 2002]) in bl2seq, which is publicly available from NCBI (ftp://ftp.ncbi.nih.gov/blast/). The default parameters of bl2seq are utilized except that filtering of low complexity regions should be turned off.

Polypeptide sequence identity may also be calculated over the entire length of the overlap between a candidate and subject polypeptide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Another method for calculating polypeptide % sequence identity is based on aligning sequences to be compared using Clustal X (Jeanmougin et al., 1998, Trends Biochem. Sci. 23, 403-5.)

Polypeptide variants as disclosed herein also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [November 2002]) from NCBI (ftp://ftp.ncbi.nih.gov/blast/). The similarity of polypeptide sequences may be examined using the following unix command line parameters:
bl2seq-i peptideseq1-j peptideseq2-F F-p blastp

The parameter-F F turns off filtering of low complexity sections. The parameter-p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Variant polypeptide sequences preferably exhibit an E value of less than 1×10⁻¹⁰ more preferably less than 1×10⁻²⁰, more preferably less than 1×10⁻³⁰, more preferably less than 1×10⁻⁴⁰, more preferably less than 1×10⁻⁵⁰, more preferably less than 1×10⁻⁶⁰, more preferably less than 1×10⁻⁷⁰, more preferably less than 1×10⁻⁸⁰, more preferably less than 1×10⁻⁹⁰ and most preferably less than 1×10⁻¹⁰⁰ when compared with any one of the specifically identified sequences.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

The function of a variant polypeptide disclosed herein as modifier of GGP translation, production and/or activity may be assessed for example by expressing such a sequence in a host cell and testing its activity as described herein in the Examples. Function of a variant may also be tested for its ability to alter AsA content in plants, also as described in the Examples section herein.

In various embodiments the polypeptide or polypeptide variant may comprise one or more SANT/MYB domains. A SANT domain is a protein domain that allows many chromatin remodelling proteins to interact with histones (Boyer et al., 2002, Molecular Cell 10 (4): 935-942; Boyer et al., 2004, Nature Reviews Molecular Cell Biology 5 (2): 158-163). SANT domains have an acidic predicted isoelectric point (pI), whereas MYB domains have a basic pI (Ko et al., 2008, Molecular Cancer 7 (1): 77). MYBS1 proteins are predicted to have two SANT/MYB domains. For example, MYBS1 from Actinidia eriantha (SEQ ID No: 103) has an N-terminal SANT domain with a predicted acidic isoelectric point (pI), and a second SANT/MYB region with a predicted basic pI.

### 6. Methods for Identifying Variants

### Physical Methods

Variant polypeptides may be identified using PCR-based methods (Mullis et al., Eds. 1994 The Polymerase Chain Reaction, Birkhauser). Typically, the polynucleotide sequence of a primer, useful to amplify variants of polynucleotide molecules as disclosed herein by PCR, may be based on a sequence encoding a conserved region of the corresponding amino acid sequence.

Alternatively library screening methods, well known to those skilled in the art, may be employed (Sambrook et al., Molecular Cloning: A Laboratory Manual, 2nd Ed. Cold Spring Harbor Press, 1987). When identifying variants of the probe sequence, hybridization and/or wash stringency will typically be reduced relatively to when exact sequence matches are sought.

Polypeptide variants may also be identified by physical methods, for example by screening expression libraries using antibodies raised against polypeptides disclosed herein (Sambrook et al., Molecular Cloning: A Laboratory Manual, 2nd Ed. Cold Spring Harbor Press, 1987) or by identifying polypeptides from natural sources with the aid of such antibodies.

### Computer Based Methods

The variant sequences as disclosed herein, including both polynucleotide and polypeptide variants, may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29:1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [November 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, MD 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, including BLASTN, BLASTP, and BLASTX, is described in the publication of Altschul et al., Nucleic Acids Res. 25:3389-3402, 1997.

The “hits” to one or more database sequences by a queried sequence produced by BLASTN, BLASTP, BLASTX, tBLASTN, BLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680) CLUSTAL Omega (Sievers et al., (2011). Molecular Systems Biology 7:539, https://www.ebi.ac.uk/Tools/msa/clustalo/) or T-COFFEE (Cedric Notredame, Desmond G. Higgins, Jaap Heringa, T-Coffee: A novel method for fast and accurate multiple sequence alignment, J. Mol. Biol. (2000) 302:205-217)) or PILEUP, which uses progressive, pairwise alignments. (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

Another example of a protein domain model database is Pfam (Sonnhammer et al., 1997, A comprehensive database of protein families based on seed alignments, Proteins, 28:405-420; Finn et al., 2010, The Pfam protein families database′, Nucl. Acids Res., 38: D211-D222). “Pfam” refers to a large collection of protein domains and protein families maintained by the Pfam Consortium and available at several sponsored world wide web sites, including: pfam.xfam.org/(European Bioinformatics Institute (EMBL-EBI). The latest release of Pfam is Pfam 35.0 (November 2021). Pfam domains and families are identified using multiple sequence alignments and hidden Markov models (HMMs). Pfam-A family or domain assignments, are high quality assignments generated by a curated seed alignment using representative members of a protein family and profile hidden Markov models based on the seed alignment. (Unless otherwise specified, matches of a queried protein to a Pfam domain or family are Pfam-A matches.) All identified sequences belonging to the family are then used to automatically generate a full alignment for the family (Sonnhammer (1998) Nucleic Acids Research 26, 320-322; Bateman (2000) Nucleic Acids Research 26, 263-266; Bateman (2004) Nucleic Acids Research 32, Database Issue, D138-D141; Finn (2006) Nucleic Acids Research Database Issue 34, D247-251; Finn (2010) Nucleic Acids Research Database Issue 38, D21 1-222). By accessing the Pfam database, for example, using the above-referenced website, protein sequences can be queried against the HMMs using HMMER homology search software {e.g., HMMER2, HMMER3, or a higher version, hmmer.org). Significant matches that identify a queried protein as being in a pfam family (or as having a particular Pfam domain) are those in which the bit score is greater than or equal to the gathering threshold for the Pfam domain. Expectation values (e values) can also be used as a criterion for inclusion of a queried protein in a Pfam or for determining whether a queried protein has a particular Pfam domain, where low e values (much less than 1.0, for example less than 0.1, or less than or equal to 0.01) represent low probabilities that a match is due to chance.

### 7. Methods for Isolating or Producing Polynucleotides

The polynucleotide molecules disclosed herein can also be isolated by using a variety of techniques known to those of ordinary skill in the art. By way of example, such polynucleotides can be isolated through use of the polymerase chain reaction (PCR) described in Mullis et al., Eds. 1994 The Polymerase Chain Reaction, Birkhauser, incorporated herein by reference. The polynucleotides as herein disclosed can be amplified using primers, as defined herein, derived from the polynucleotide sequences as herein disclosed.

Further methods for isolating polynucleotides as disclosed herein include use of all, or portions of, the polynucleotides having the sequence set forth herein as hybridization probes. The technique of hybridizing labelled polynucleotide probes to polynucleotides immobilized on solid supports such as nitrocellulose filters or nylon membranes, can be used to screen the genomic or cDNA libraries. Exemplary hybridization and wash conditions are: hybridization for 20 hours at 65° C. in 5.0×SSC, 0.5% sodium dodecyl sulfate, 1×Denhardt's solution; washing (three washes of twenty minutes each at 55° C.) in 1.0×SSC, 1% (w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

The polynucleotide fragments as disclosed herein may be produced by techniques well-known in the art such as restriction endonuclease digestion, oligonucleotide synthesis and PCR amplification.

A partial polynucleotide sequence may be used, in methods well-known in the art to identify the corresponding full-length polynucleotide sequence. Such methods include PCR-based methods, 5′RACE (Frohman M A, 1993, Methods Enzymol. 218:340-56) and hybridization-based method, computer/database-based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

It may be beneficial, when producing a transgenic plant from a particular species, to transform such a plant with a sequence or sequences derived from that species. The benefit may be to alleviate public concerns regarding cross-species transformation in generating transgenic organisms. Additionally when down-regulation of a gene is the desired result, it may be necessary to utilise a sequence identical (or at least highly similar) to that in the plant, for which reduced expression is desired. For these reasons among others, it is desirable to be able to identify and isolate orthologues of a particular gene in several different plant species.

Variants (including orthologues) may be identified by the methods described herein.

### 8. Methods for Isolating or Producing Polypeptides

The polypeptides as disclosed herein, including variant polypeptides, may be prepared using peptide synthesis methods well known in the art such as direct peptide synthesis using solid phase techniques (e.g. Stewart et al., 1969, in Solid-Phase Peptide Synthesis, WH Freeman Co, San Francisco California), or automated synthesis, for example using an Applied Biosystems 431A Peptide Synthesizer (Foster City, California). Mutated forms of the polypeptides may also be produced during such syntheses.

The polypeptides and variant polypeptides as disclosed herein may also be purified from natural sources using a variety of techniques that are well known in the art (e.g. Deutscher, 1990, Ed, Methods in Enzymology, Vol. 182, Guide to Protein Purification,).

Alternatively the polypeptides and variant polypeptides as disclosed herein may be expressed recombinantly in suitable host cells as disclosed herein and separated from the cells as discussed below.

### 9. Constructs, Vectors and Components Thereof

According to one embodiment, the polynucleotides useful in the methods according to some embodiments of the invention may be provided in a nucleic acid construct useful in transforming a plant or host cell. Suitable plant and host cells are described herein.

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a synthetic or recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary regulatory elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be expressed, and
c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

Because many microorganisms are capable of expressing multiple gene products from a polycistronic mRNA, multiple polypeptides can be expressed under the control of a single regulatory region for those microorganisms, if desired.

“Operably-linked” means that the sequence to be expressed is placed under the control of regulatory elements that include promoters, tissue-specific regulatory elements, temporal regulatory elements, enhancers, repressors and terminators. Typically, the translation initiation site of the translational reading frame of the coding sequence is positioned between one and about fifty nucleotides downstream of the regulatory region for a monocistronic gene.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also can include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region can be present, e.g., introns, enhancers, upstream activation regions, transcription terminators, and inducible elements.

The term “noncoding region” includes to untranslated sequences that are upstream of the translational start site and downstream of the translational stop site. These sequences are also referred to respectively as the 5′ UTR and the 3′ UTR. These sequences may include elements required for transcription initiation and termination and for regulation of translation efficiency. The term “noncoding” also includes intronic sequences within genomic clones.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to nontranscribed cis-regulatory elements upstream of the coding region that regulate gene transcription. Promoters comprise cis-initiator elements which specify the transcription initiation site and conserved boxes such as the TATA box, and motifs that are bound by transcription factors.

A “transgene” is a polynucleotide that is taken from one organism and introduced into a different organism by transformation. The transgene may be derived from the same species or from a different species as the species of the organism into which the transgene is introduced.

An “inverted repeat” is a sequence that is repeated, where the second half of the repeat is in the complementary strand, e.g.,

(5′)GATCTA . . . TAGATC(3′)
(3′)CTAGAT . . . ATCTAG(5′)

Read-through transcription will produce a transcript that undergoes complementary base-pairing to form a hairpin structure provided that there is a 3-5 bp spacer between the repeated regions.

### 10. Methods for Producing Constructs and Vectors

The genetic constructs as disclosed herein comprise one or more polynucleotide sequences as disclosed herein and/or polynucleotides encoding polypeptides as disclosed herein, and may be useful for transforming, for example, bacterial, fungal, insect, mammalian or plant organisms. The genetic constructs disclosed herein are intended to include expression constructs as herein defined.

Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

### 11. Host Cells

In other embodiments, there is provided a host cell which comprises a genetic construct or vector as disclosed herein. In preferred embodiments, the host cell is genetically modified to express one of:
a) a polynucleotide encoding a polypeptide with the amino acid sequence of any one of SEQ ID NO: 1-102 and 348-356, or a variant of the polypeptide, or
b) a polynucleotide comprising a nucleotide sequence selected from any one of the sequences SEQ ID NO: 103-108, or a variant thereof, and/or, one of
c) a polynucleotide encoding a polypeptide with the amino acid sequence of any one of SEQ ID NO: 109 to 209, or a variant of the polypeptide, or
d) a polynucleotide comprising a nucleotide sequence of SEQ ID NO: 210, or a variant thereof.

Host cells comprising genetic constructs, such as expression constructs, as disclosed herein are useful in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides disclosed herein. Such methods may involve the culture of host cells in an appropriate medium in conditions suitable for or conducive to expression of a polynucleotide or polypeptide disclosed herein. The expressed recombinant polypeptide, which may optionally be secreted into the culture, may then be separated from the medium, host cells or culture medium by methods well known in the art (e.g. Deutscher, Ed, 1990, Methods in Enzymology, Vol 182, Guide to Protein Purification).

### 12. Methods for Producing Plant Cells and Plants Comprising Constructs and Vectors

In other embodiments there is provided a plant cell which comprises a genetic construct as disclosed herein, and a plant cell modified to alter expression of a polynucleotide or polypeptide as disclosed herein. Plants comprising such cells are also provided.

Alteration of GGP translation, production and/or activity may be altered in a plant through methods according to some embodiments of the invention. Such methods may involve the transformation of plant cells and plants, with a construct designed to alter expression of a polynucleotide or polypeptide that alters GGP expression, and/or AsA content in such plant cells and plants. Such methods also include the transformation of plant cells and plants with a combination of a construct as disclosed herein and one or more other constructs designed to alter expression of one or more polynucleotides or polypeptides which modulate GGP activity and/or AsA content in such plant cells and plants.

Methods for transforming plant cells, plants and portions thereof with polypeptides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin.; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

Transformation may be transient or stable, as is known in the art. Transient transformation results in the temporary introduction of nucleic acid into a cell, however the introduced nucleic acid does not integrate into the cell's genome. Stable transformation results in modification of the cell's genome, which will persist and may be passed on to subsequent generations of the cell. Preferably the plant cells and plants are stably transformed.

In some embodiments, the plant cell to be transformed comprises an endogenous GGP gene, preferably a functional GGP gene. For example, in some embodiments, the plant cell to be transformed has been previously transformed with a functional GGP gene. In other embodiments, the plant cell is co-transformed or subsequently transformed with a functional GGP gene. A functional GGP gene is a gene that encodes and expresses a functional GGP protein. A functional GGP protein is one that is capable of performing one or more functions of a GGP protein, such as catalysing the conversion of GDP-L-galactose to L-galactose 1-phosphate.

In some embodiments the GGP gene is a GGP3 gene. In some embodiments, the GGP gene encodes a protein with at least about 70% sequence identity to SEQ ID No: 365, preferably at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, at least about 99%, or 100% sequence identity to SEQ ID No: 365.

In some embodiments, the GGP gene has a promoter that comprises one or more MYBS1 binding sites, preferably two or more MYBS1 binding sites. In some embodiments, the methods described herein may further comprise gene-editing the promoter of an endogenous GGP gene to provide a promoter comprising one or more MYBS1 binding sites. This may be achieved, for example, by modifying an endogenous GGP promoter. Alternatively, this may be achieved by replacing an endogenous promoter with a GGP promoter comprising one or more MYBS1 binding sites, such as SEQ ID No: 345. In some embodiments, the methods described herein may further comprise transforming the plant cell with a GGP gene. In some embodiments, the methods may further comprise transforming the plant cell with a GGP gene having a promoter that comprises one or more MYBS1 binding sites, preferably two or more MYBS1 binding sites.

An MYBS1 binding site is any nucleotide sequence that an MYBS1 protein is capable of binding to. MYBS1 binding sites may be determined by a number of methods known in the art, for example using bioinformatic prediction (using, for example, JASPAR 2020 (Fornes et al., 2019, Nucleic Acids Research 48 (D1): D87-D92)) or using an electrophoretic mobility shift assay (EMSA). Some exemplary methods for identifying MYBS1 binding sites are presented in Example 4.

Preferably the one or more MYBS1 binding sites comprise the sequence TCTTATC or its reverse complement GATAAGA. In some embodiments, the GGP promoter has at least about 70% sequence identity to SEQ ID No: 345, preferably at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, at least about 99%, or 100% sequence identity to SEQ ID No: 345.

### Methods for Genetic Manipulation of Plants

A number of plant transformation strategies are available (e.g. Birch, 1997, Ann Rev Plant Phys Plant Mol Biol, 48, 297, Hellens R P, et al (2000) Plant Mol Biol 42:819-32, Hellens R et al Plant Meth 1:13). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species.

Genetic constructs for expression of genes in transgenic plants typically include promoters for driving the expression of one or more cloned polynucleotide, terminators and selectable marker sequences to detect presence of the genetic construct in the transformed plant.

The promoters suitable for use in the constructs as described herein are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences as herein disclosed. Examples of constitutive plant promoters include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are described in the scientific literature. Exemplary promoters are described, e.g., in WO 02/00894, which is herein incorporated by reference.

In some embodiments, the promoter is active in leaves and/or roots. In some embodiments, the promoter is a tissue-specific promoter that is active in leaves and/or roots. In a preferred embodiment, the promoter is active in leaves. Promoters that are active in leaves and/or roots, including tissue-specific promoters, are known in the art.

Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phosphotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Use of genetic constructs comprising reporter genes (coding sequences which express an activity that is foreign to the host, usually an enzymatic activity and/or a visible signal (e.g., luciferase, GUS, GFP) which may be used for promoter expression analysis in plants and plant tissues are also contemplated. The reporter gene literature is reviewed in Herrera-Estrella et al., 1993, Nature 303, 209, and Schrott, 1995, In: Gene Transfer to Plants (Potrykus, T., Spangenberg. Eds) Springer Verlag. Berline, pp. 325-336.

The following are representative publications disclosing genetic transformation protocols that can be used to genetically transform the following plant species: Rice (Alam et al., 1999, Plant Cell Rep. 18, 572); apple (Yao et al., 1995, Plant Cell Reports 14, 407-412); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9,: 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci. 104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat.Nos. 5,591,616 and 6,037,522); brassica (U.S. Pat. Nos. 5,188,958; 5,463,174 and 5,750,871); cereals (U.S. Pat. No. 6,074,877); pear (Matsuda et al., 2005, Plant Cell Rep. 24 (1): 45-51); Prunus (Ramesh et al., 2006 Plant Cell Rep. 25 (8): 821-8; Song and Sink 2005 Plant Cell Rep. 2006; 25 (2): 117-23; Gonzalez Padilla et al., 2003 Plant Cell Rep. 22 (1): 38-45); strawberry (Oosumi et al., 2006 Planta. 223 (6): 1219-30; Folta et al., 2006 Planta April 14; PMID: 16614818), rose (Li et al., 2003), Rubus (Graham et al., 1995 Methods Mol Biol. 1995; 44:129-33), tomato (Dan et al., 2006, Plant Cell Reports V25: 432-441), apple (Yao et al., 1995, Plant Cell Rep. 14, 407-412) and Actinidia eriantha (Wang et al., 2006, Plant Cell Rep. 25,5:425-31). Transformation of other species is also contemplated by the invention. Suitable methods and protocols are available in the scientific literature.

In one embodiment, there is provided a method of producing a plant cell or plant with increased L-ascorbic acid (AsA) content or increased GDP-L-galactose phosphorylase 3 (GGP) translation, production and/or activity, the method comprising upregulating in the plant cell or plant expression of one of:
a) a polynucleotide encoding a polypeptide with the amino acid sequence of any one of SEQ ID NO: 1-102 and 348-356, or a variant of the polypeptide, or
b) a polynucleotide comprising a nucleotide sequence selected from any one of the sequences SEQ ID NO: 103-108, or a variant thereof, and/or, one of
c) a polynucleotide encoding a polypeptide with the amino acid sequence of any one of SEQ ID NO: 109 to 209, or a variant of the polypeptide, or
d) a polynucleotide comprising a nucleotide sequence of SEQ ID NO: 210, or a variant thereof.

Several methods known in the art may be employed to alter expression of a nucleotide and/or polypeptide as herein disclosed. Such methods include but are not limited to Tilling (Till et al., 2003, Methods Mol Biol, 2%, 205), and so called “Deletagene” technology (Li et al., 2001, Plant Journal 27 (3), 235)

Other methods may involve the use of sequence-specific nucleases that generate targeted double-stranded DNA breaks in genes of interest. Examples of such methods include: zinc finger nucleases (Curtin, et al., 2011, Sander, et al., 2011), transcription activator-like effector nucleases or “TALENs” (Cermak, et al., 2011, Mahfouz, et al., 2011, Li, et al., 2012), and LAGLIDADG homing endonucleases, also termed “meganucleases” (Tzfira, et al., 2012).

Targeted genome editing using engineered nucleases such as clustered, regularly interspaced, short palindromic repeat (CRISPR) technology, is an important new approach for generating RNA-guided nucleases, such as Cas9, with customizable specificities. Genome editing mediated by these nucleases has been used to rapidly, easily and efficiently modify endogenous genes in a wide variety of biomedically important cell types and in organisms that have traditionally been challenging to manipulate genetically. A modified version of the CRISPR-Cas9 system has been developed to recruit heterologous domains that can regulate endogenous gene expression or label specific genomic loci in living cells (Sander and Joung, 2014). The technique is applicable to fungi (Nodvig, et al., 2015).

Upregulating expression of a polypeptide in a plant, for example by genome editing, can be achieved by: (i) replacing an endogenous sequence encoding the polypeptide of interest or a regulatory sequence under the control which it is placed, and/or (ii) inserting a new gene encoding the polypeptide of interest in a targeted region of the genome, and/or (iii) introducing point mutations which result in up-regulation of the endogenous gene encoding the polypeptide of interest (e.g., by altering the regulatory sequences such as promoter, enhancers, 5′-UTR and/or 3′-UTR, or mutations in the coding sequence).

In this manner, an endogenous gene encoding a polypeptide with the amino acid sequence of any one of SEQ ID NO: 1-102 and 348-356 or 109-209 or a variant of the polypeptide, or comprising a nucleotide sequence selected from any one of the sequences SEQ ID NO: 103-108 or 210 or a variant thereof, may be upregulated, resulting in increased AsA content and/or increased GGP translation, production and/or activity.

Antibodies or fragments thereof, targeted to a particular polypeptide may also be expressed in plants to modulate the activity of that polypeptide (Jobling et al., 2003, Nat. Biotechnol., 21 (1), 35). Transposon tagging approaches may also be applied. Additionally peptides interacting with a polypeptide as herein disclosed may be identified through technologies such as phage-display (Dyax Corporation). Such interacting peptides may be expressed in or applied to a plant to affect activity of a polypeptide as herein disclosed. Use of each of the above approaches in alteration of expression of a nucleotide and/or polypeptide as herein disclosed is specifically contemplated.

The terms “to alter expression of” and “altered expression” of a polynucleotide or polypeptide as herein disclosed, are intended to encompass the situation where genomic DNA corresponding to a polynucleotide as herein disclosed is modified thus leading to altered expression of a polynucleotide or polypeptide as herein disclosed. Modification of the genomic DNA may be through genetic transformation or other methods known in the art for inducing mutations. The “altered expression” can be related to an increase or decrease in the amount of messenger RNA and/or polypeptide produced and may also result in altered activity of a polypeptide due to alterations in the sequence of a polynucleotide and polypeptide produced.

### 13. Methods of Selecting Plants

Methods are also provided for selecting plants with altered GGP activity or AsA content. Such methods involve testing of plants for altered expression of a polynucleotide or polypeptide as herein disclosed. Such methods may be applied at a young age or early developmental stage when the altered GGP activity or AsA content may not necessarily be easily measurable.

The expression of a polynucleotide, such as a messenger RNA, is often used as an indicator of expression of a corresponding polypeptide. Exemplary methods for measuring the expression of a polynucleotide include but are not limited to Northern analysis, RT-PCR and dot-blot analysis (Sambrook et al., Molecular Cloning: A Laboratory Manual, 2nd Ed. Cold Spring Harbor Press, 1987). Polynucleotides or portions of the polynucleotides as herein disclosed are thus useful as probes or primers, as herein defined, in methods for the identification of plants with altered levels GGP or AsA. The polynucleotides as herein disclosed may be used as probes in hybridization experiments, or as primers in PCR based experiments, designed to identify such plants.

Alternatively antibodies may be raised against polypeptides as herein disclosed. Methods for raising and using antibodies are standard in the art (see for example: Antibodies, A Laboratory Manual, Harlow A Lane, Eds, Cold Spring Harbour Laboratory, 1998). Such antibodies may be used in methods to detect altered expression of the polypeptides disclosed herein. Such methods may include ELISA (Kemeny, 1991, A Practical Guide to ELISA, NY Pergamon Press) and Western analysis (Towbin & Gordon, 1994, J Immunol Methods, 72, 313).

These approaches for analysis of polynucleotide or polypeptide expression and the selection of plants with altered GGP activity or altered AsA content are useful in conventional breeding programs designed to produce varieties with altered GGP activity or AsA content.

### 14. Plants

The term “plant” is intended to include a whole plant, any part of a plant, propagules and progeny of a plant.

The term “propagule” means any part of a plant that may be used in reproduction or propagation, either sexual or asexual, including seeds and cuttings.

A “transgenic” or “transformed” plant refers to a plant which contains new genetic material as a result of genetic manipulation or transformation. The new genetic material may be derived from a plant of the same species as the resulting transgenic or transformed plant or from a different species. A transformed plant includes a plant which is either stably or transiently transformed with new genetic material. Preferably a transformed plant is stably transformed.

The plants and plant cells according to some embodiments of the invention may be grown and either self-ed or crossed with a different plant strain and the resulting hybrids, with the desired phenotypic characteristics, may be identified. Two or more generations may be grown to ensure that the subject phenotypic characteristics are stably maintained and inherited. Plants and plant cells resulting from such standard breeding approaches also form an aspect of the present invention.

The function of a variant polynucleotide disclosed herein as encoding a MYBS1-like or GBF3-like transcription factor may be assessed for example by expressing such a sequence in bacteria and testing activity of the encoded protein as described in the Example section herein.

Alteration of GGP activity and/or AsA content may also be altered in a plant or plant cell through methods according to some embodiments of the invention. Such methods may involve the transformation of plant cells and plants, with a construct as herein disclosed designed to alter expression of a polynucleotide or polypeptide which modulates GGP activity and/or AsA content in such plant cells and plants. Such methods preferably also include the transformation of plant cells and plants with a combination of the construct as herein disclosed and one or more other constructs designed to alter expression of one or more other polynucleotides or polypeptides which modulate AsA content in such plant cells and plants.

Any plant is suitable for use in the invention. The L-galactose biosynthetic pathway, which produces AsA is present in all plants. The enzyme GDP-L-galactose phosphorylase (GGP) is critical to the pathway and is also present in all plants. Therefore, the methods of the invention can be used to alter GGP expression and/or increase AsA production in any plant.

In various embodiments the plant or plant cell is a gymnosperm plant species.

In a further embodiment the plant or plant cell is an angiosperm plant species.

In a further embodiment the plant or plant cell is a dicotyledonous plant species.

Plants and plant cells that are particularly useful in the methods of the invention disclosed herein include all plants and plant cells which belong to the superfamily Viridiplantae, in particular monocotyledonous and dicotyledonous plants including a fodder or forage legume, ornamental plant, food crop, tree, or shrub selected from the list comprising Abrus precatorius, Acacia spp., Acer spp., Actinidia spp., Aesculus spp., Arachis duranensis, Arabidopsis spp., Arachis ipaensis, Betula spp., Brassica spp., Buddleja alternifolia, Cajanus cajan, Camellia sinensis, Capsicum spp., Carex littledalei, Carica papaya, Carya illinoinensis, Castanea mollissima, Catharanthus roseus, Cephalotus follicularis, Chenopodium quinoa, Cinnamomum cassia, Citrus clementina, Citrus sinensis, Citrus unshiu, Coffea arabica, Coronillia varia, Corchorus olitorius, Corylus heterophyll, Cotoneaster serotina, Crataegus spp., Cucumis spp., Cupressus spp., Cyathea dealbata, Cydonia oblonga, Cryptomeria japonica, Cymbopogon spp., Dalbergia monetaria, Davallia divaricata, Desmodium spp., Dicksonia squarosa, Diheteropogon amplectens, Dioclea spp, Dolichos spp., Dorycnium rectum, Durio zibethinus, Echinochloa pyramidalis, Ehrartia spp., Eleusine coracana, Eragrestis spp., Erythrina spp., Eucalyptus spp., Euclea schimperi, Eulalia villosa, Fagopyrum spp., Feijoa sellowiana, Fragaria spp., Flemingia spp, Freycinetia banksii, Geranium thunbergii, Ginkgo biloba, Glycine javanica, Glycine max, Glycine soja, Gliricidia spp, Gossypium anomalum, Gossypium barbadense, Gossypium darwinii, Gossypium hirsutum, Gossypium mustelinum, Gossypium raimondii, Gossypium stocksii, Grevillea spp., Guibourtia coleosperma, Hedysarum spp., Helianthus annuus, Hemarthia altissima, Herrania umbratical, Heteropogon contortus, Hevea brasiliensis, Hibiscus syriacus, Hordeum vulgare, Hyparrhenia rufa, Hypericum erectum, Hyperthelia dissoluta, indigo incarnata, Ipomoea nil, Ipomoea triloba, Iris spp., Jatropha curcas, Juglans macrocarpa, Juglans regia, Juglans macrocarpa x Juglans regia, Lactuca saligna, Lactuca sativa, Leptarrhena pyrolifolia, Lespediza spp., Lettuca spp., Leucaena leucocephala, Loudetia simplex, Lotonus bainesii, Lotus spp., Lupinus angustifolius, Macadamia integrifolia, Macrotyloma axillare, Malus spp., Manihot esculenta, Medicago sativa, Metasequoia glyptostroboides, Morella rubra, Morus notabilis, Mucuna pruriens, Musa sapientum, Nicotiana spp., Nelumbo nucifera, Nyssa sinensis, Onobrychis spp., Ornithopus spp., Oryza spp., Panicum hallii, Panicum virgatum, Papaver somniferum, Peltophorum africanum, Pennisetum spp., Persea gratissima, Petunia spp., Phalaenopsis equestris, Phaseolus spp., Phoenix canariensis, Phoenix dactylifera, Phormium cookianum, Photinia spp., Picea glauca, Pinus spp., Pisum sativum, Podocarpus totara, Pogonarthria fleckii, Pogonarthria squarrosa, Populus spp., Prosopis alba, Prosopis cineraria, Prunus armeniaca, Prunus avium, Prunus dulcis, Prunus mume, Prunus persica Prunus yedoensis var. nudiflora, Pseudotsuga menziesii, Pterolobium stellatum, Punica granatum, Pyrus spp., Quercus spp., Rhamnella rubrinervis, Rhaphiolepsis umbellata, Rhodamnia argentea, Rhododendron griersonianum, Rhododendron simsii, Rhododendron williamsianum, Rhopalostylis sapida, Rhus natalensis, Ribes grossularia, Ribes spp., Ricinus communis, Robinia pseudoacacia, Rosa spp., Rubus spp., Salix spp., Schyzachyrium sanguineum, Sciadopitys verticillata, Sequoia sempervirens, Sequoiadendron giganteum, Solanum chilense, Solanum commersonii, Solanum lycopersicum, Solanum pennellii, Solanum tuberosum, Sorghum bicolor, Spatholobus suberectus, Spinacia spp., Sporobolus fimbriatus, Stiburus alopecuroides, Stylosanthos humilis, Tadehagi spp, Taxodium distichum, Telopea speciosissima, Trema orientale, Tetracentron sinense, Themeda triandra, Theobroma cacao, Trema orientale, Trifolium spp., Triticum spp., Tsuga heterophylla, Vaccinium spp., Vicia spp., Vigna angularis, Vigna radiata var. radiata, Vigna unguiculate, Vitis riparia, Vitis vinifera, Watsonia pyramidata, Zantedeschia aethiopica, Zea mays, Ziziphus jujuba, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash and tea, amongst others.

In some embodiments, the plant or plant cell may be a crop plant, such as a food crop or a biofuel crop. In one embodiment, the plant or plant cell may be a cereal, legume, or fruit plant. In one embodiment, the plant or plant cell may be a cereal or legume plant. In one embodiment, the plant or plant cell may be from the family Poaceae. In one embodiment, the cereal may be rice, wheat, oats, barley, triticale, rye, finger millet, Sonoran millet, sorghum, or maize. In one embodiment, the cereal may be barley, wheat, rice, or maize. In another embodiment, the legume may be alfalfa or soybeans. In another embodiment, the fruit plant or plant cell may be apple, kiwifruit, or tomato. In one embodiment, the fruit plant or plant cell may be apple or kiwifruit. In one embodiment, the plant or plant cell may be selected from the group comprising kiwifruit, maize, tomato, wheat, barley, tobacco, soybean, rice, apple, cotton, brassicas, and alfalfa. In one embodiment, the plant or plant cell may be selected from the list consisting of Actinidia arguta, Actinidia chinensis, Actinidia eriantha, Arabidopsis thaliana, Glycine max, Gossypium hirsutum, Hordeum vulgare, Malus domestica, Medicago sativa, Nicotiana benthamiana, Nicotiana tabacum, Oryza sativa, Solanum lycopersicum, Triticum aestivum, and Zea mays. In one embodiment, the plant or plant cell may be a rice, soybean, or kiwifruit plant or plant cell. In one embodiment, the plant or plant cell may be selected from the list consisting of Actinidia arguta, Actinidia chinensis, Actinidia eriantha, Glycine max, and Oryza sativa.

In some embodiments, plants or plant cells grown specifically for “biomass” may be used. For example, suitable plants or plant cells include corn, switchgrass, sorghum, miscanthus, sugarcane, poplar, pine, wheat, rice, soy, cotton, barley, turf grass, tobacco, potato, bamboo, rape, sugar beet, sunflower, willow, and eucalyptus. In further embodiments, the plant or plant cell is switchgrass (Panicum virgatum), giant reed (Arundo donax), reed canarygrass (Phalaris arundinacea), Miscanthusxgiganteus, Miscanthus sp., sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, or Kentucky bluegrass amongst others.

Alternatively algae and other non-Viridiplantae can be used for the methods of some embodiments of the invention.

In some embodiments the plant or plant cell is a fruit species. In one embodiment, the plant or plant cell is a plant of the Cucurbitaceae family, such as S. grosvenorii. In various embodiments the fruit species is selected from the group comprising the following genera: Actinidia, Malus, Citrus, Fragaria and Vaccinium.

In a further embodiment the plant or plant cell is selected from the group consisting of Actinidia eriantha, Cucumis sativus, Glycine max, Solanum lycopersicum, Vitis vinifera, Arabidopsis thaliana, Malus x domesticus, Medicago truncatula, Populus trichocarpa, Actinidia arguta, Actinidia chinensis, Fragaria vulgaris, Solanum tuberosum, and Zea mays. In a further embodiment the plant or plant cell is selected from the group consisting of Actinidia eriantha, Cucumis sativus, Glycine max, Vitis vinifera, Arabidopsis thaliana, Malus x domesticus, Medicago truncatula, Populus trichocarpa, Actinidia arguta, Actinidia chinensis, Fragaria vulgaris, and Zea mays.

According to one embodiment, the plant or plant cell is a plant of the Rosaceae family, such as but not limited to, apple tree, pear tree, quince tree, apricot tree, plum tree, cherry tree, peach tree, raspberry bush, loquat tree, strawberry plant, almond tree, and ornamental trees and shrubs (e.g. roses, meadowsweets, photinias, firethorns, rowans, and hawthorns).

In one embodiment the pear is of the genus Pyrus. Preferred pear species include: Pyrus calleryana, Pyrus caucasica, Pyrus communis, Pyrus elaeagrifolia, Pyrus hybrid cultivar, Pyrus pyrifolia, Pyrus salicifolia, Pyrus ussuriensis and Pyrus x bretschneideri.

In one embodiment the plant or plant cell is of the genus Malus. Preferred Malus species include: Malus aldenhamensis, Malus angustifolia, Malus asiatica, Malus baccata, Malus coronaria, Malus domestica, Malus doumeri, Malus florentina, Malus floribunda, Malus fusca, Malus halliana, Malus honanensis, Malus hupehensis, Malus ioensis, Malus kansuensis, Malus mandshurica, Malus micromalus, Malus niedzwetzkyana, Malus ombrophilia, Malus orientalis, Malus prattii, Malus prunifolia, Malus pumila, Malus sargentii, Malus sieboldii, Malus sieversii, Malus sylvestris, Malus toringoides, Malus transitoria, Malus trilobata, Malus tschonoskii, Malus x domestica, Malus x domestica x Malus sieversii, Malus x domestica x Pyrus communis Malus xiaojinensis, Malus yunnanensis, Malus sp., and Mespilus germanica. In one embodiment the plant species is Malus domestica. In a specific embodiment, the plant is a Malus domestica, Malus trilobata or Malus sieboldii.

In another embodiment, the plant or plant cell is a plant of a Vitis species. Exemplary Vitis species include, but are not limited to, Vitis piasezkii maxim and Vitis saccharifera makino.

In one embodiment the plant or plant cell is a plant from a species selected from a group comprising but not limited to the following genera: Smilax (eg Smilax glyciphylla), and Fragaria.

In a further embodiment the plant or plant cell is from a vegetable species selected from a group comprising but not limited to the following genera: Brassica, Lycopersicon and Solanum.

Particularly preferred vegetable plant species are: Solanum lycopersicum (formerly Lycopersicon esculentum) and Solanum tuberosum.

In a further embodiment the plant or plant cell is from monocotyledonous species.

In a further embodiment the plant or plant cell is from a crop species selected from a group comprising but not limited to the following genera: Glycine, Zea, Hordeum and Oryza. Particularly preferred crop plant species are: Oryza sativa, Glycine max and Zea mays.

In various embodiments, the plant or plant cell is from a food crop or biofuel crop. Species useful for food or biofuel production are known in the art. For example, species useful for biofuel production may include Miscanthus x giganteus, Cenchrus purpureus, Cocos nucifera L., Jatropha L., and Ricinus communis L.

In various embodiments, the plant or plant cell is selected from the group consisting of rice, wheat, oats, barley, triticale, rye, finger millet, Sonoran millet, sorghum, maize, banana, Miscanthus, elephant grass or Uganda grass, coconut, sugarcane, cotton, sunflower, soybean, flax, sesame, jatropha, sugar beet, alfalfa, forage brassica, oilseed rape, mustard seed, almond, walnut, pecan, macadamia, peanut, and castor bean.

Plants may be grouped by phylogenetic classification as described in APG IV (The Angiosperm Phylogeny Group et al. (2016), An update of the Angiosperm Phylogeny Group classification for the orders and families of flowering plants: APG IV, Botanical Journal of the Linnean Society 181 (1), 1-20, https://doi.org/10.1111/boj 12385), which is incorporated herein by reference.

In a further embodiment the plant or plant cell is selected from the group consisting of magnoliids, monocots, basal eudicots, Gunnerales, Dilleniales, superrosids, Santalales, Berberidopsidales, Caryophyllales, Cornales, Ericales, Campanulids, Icacinales, Metteniusales, Garryales, Boraginales, Gentianales, Vahliales, and Lamiales.

In a further embodiment the plant or plant cell is selected from the group consisting of magnoliids, monocots, basal eudicots, Gunnerales, Dilleniales, superrosids, Santalales, Berberidopsidales, Caryophyllales, Cornales, Ericales, campanulids, Icacinales, Metteniusales, Garryales, Boraginales, Gentianales, Vahliales, Lamiales, Convolvulaceae, Montiniaceae, Sphenocleaceae, and Hydroleaceae.

In a further embodiment the plant or plant cell is selected from the group consisting of magnoliids, monocots, basal eudicots, Gunnerales, Dilleniales, Fabids, Geraniales, Myrtales, Crossosomatales, Picramniales, Malvales, Huerteales, Sapindales, Vitales, Saxifragales, Santalales, Berberidopsidales, Caryophyllales, Cornales, Ericales, campanulids, Icacinales, Metteniusales, Garryales, Boraginales, Gentianales, Vahliales, Lamiales, Convolvulaceae, Montiniaceae, Sphenocleaceae, and Hydroleaceae.

In a further embodiment the plant or plant cell is selected from the group consisting of magnoliids, monocots, basal eudicots, Gunnerales, Dilleniales, Fabids, Geraniales, Myrtales, Crossosomatales, Picramniales, Malvales, Huerteales, Sapindales, Vitales, Saxifragales, Santalales, Berberidopsidales, Caryophyllales, Cornales, Ericales, campanulids, Icacinales, Metteniusales, Garryales, Boraginales, Gentianales, Vahliales, Lamiales, Convolvulaceae, Montiniaceae, Sphenocleaceae, Hydroleaceae, Akaniaceae, Tropaeolaceae, Moringaceae, Caricaceae, Setchellanthaceae, Limnanthaceae, Salvadoraceae, Bataceae, Koeberliniaceae, Emblingiaceae, Pentadiplandraceae, Resedaceae, Gyrostemonaceae, Tovariaceae, Capparaceae, and Cleomaceae.

In some embodiments, the plant or plant cell contains an endogenous GGP3 gene, preferably a functional GGP3 gene. In some embodiments, the plant or plant cell has been previously transformed with a functional GGP3 gene. In some embodiments, the plant or plant cell is co-transformed or subsequently transformed with a functional GGP3 gene. In some embodiments, the GGP3 gene has a promoter comprising one or more MYBS1 binding sites, more preferably two or more MYBS1 binding sites. Preferably the MYBS1 binding sites comprise the sequence TCTTATC or its reverse complement GATAAGA.

### 15. Methods for Extracting and Measuring AsA from Plants

Methods are also provided for the production of L-ascorbic acid (AsA) by extraction of AsA from a plant of the invention. AsA may be extracted from plants as follows:

Frozen tissue samples are ground to a fine powder in a Cryomill at liquid nitrogen temperature. About 200 mg of frozen powdered tissue is then suspended in 5 volumes of 7% metaphosphoric acid containing 2 mM TCEP (Pierce), vortexed for 20 sec and incubated in a heating block for 2 h at 40° C. TCEP is used in the extraction solution, because it is more effective reducing agent under acidic conditions than DTT, ensuring that all of vitamin C is in the ascorbic acid reduced form. The extract is centrifuged at 4° C. and twenty μL of the supernatant is injected into a Rocket Column And eluted using two solvents A (0.28% o-phosphoric acid, 0.1 mM EDTA and 0.25% methanol) and B (acetonitrile). L-ascorbic acid and other compounds were eluted using a 5-min gradient to 90% B. Standards were run with every batch or 20 samples processed. AsA is calculated from the area under the absorption at 240 nm curve at ˜1 minute of elution.

This method may be up-scaled for larger scale AsA extraction using approaches well-known to those skilled in the art.

The above methods should be considered in no way limiting and suitable variations or alternatives will be apparent to those skilled in the art.

### EXAMPLE

### 1. Materials and Methods

### Plant Materials and Growth Conditions

Fruit samples were collected from field-grown plants in Hubei, China, during the 2016-2018 growing season. The AsA content in the fruits of 48 Actinidia species and 1 cross-population (A. eriantha x A. rufa) were measured during 2017-2019. Six kiwifruit taxa with >150-fold variation in AsA content were selected to study the changes in AsA during fruit development (A. eriantha, A. latifolia, A chinensis var. deliciosa, A chinensis var. chinensis, A. rufa and A cylindrica). Tissue culture materials for tobacco (Nicotiana benthamiana) and kiwifruit (A. eriantha) were grown at 23-25° C. under long-day conditions (16 h of light/8 h of darkness). Transgenic and gene-edited plants were potted and grown in Containment Glasshouse-1 at the Wuhan Botanical Garden, Chinese Academy of Sciences, Hubei, China (14 h of light/10 h of darkness, 18° C. min/30° C. max). Samples collected from individual plants were considered biological replicates.

### Illumina RNA-Seq and Transcriptome Analysis

Fruit RNAs from 20 days after fruiting (DAF20) to DAF120 were extracted separately from A. eriantha and A. rufa. RNA was extracted using RNeasy Plant Mini Kits (Qiagen, Inc., USA). Library construction and sequencing were performed at BerryGenomics (www.berrygenomics.com) using the Illumina NovaSeq 2000 platform, obtaining paired-end reads of 150 bp and generating >20 million reads per sample. Sequencing adapters and low-quality raw reads were filtered out using Trimmomatic (DOI: 10.1093/bioinformatics/btu170) with default parameters. The filtered clean reads were mapped to the Hongyang_v3 reference genome (http://kiwifruitgenome.org/) by Hisat2 (DOI: 10.1038/s41587-019-0201-4). Read counts and FPKM (fragments per kilobase of exon per million mapped fragments) of annotated genes were calculated by HTSeq (DOI: 10.1093/bioinformatics/btu638.) and Stringtie2 (DOI: 10.1038/nbt.3122), respectively. The differentially expressed genes between the kiwifruit materials with differentiation of AsA in the same development stage were identified by an R package edgeR with a p-value <0.05, and the Gene Ontology (GO) term enrichment was performed using the Gene Ontology Resource (http://geneontology.org/).

### RNA Extraction and Quantitative RT-PCR Analysis

Quantitative reverse transcription (qRT)-PCR was performed following previously described methods (Wang et al., Plant biotechnology journal 16 (8): 1424-1433.). Total RNA was isolated with an RNA Extraction Kit (TIANGEN, Beijing, China) and the single-stranded cDNA of all samples was obtained using a one-step gDNA removal and cDNA Synthesis Supermix Kit (TransGen, Beijing, China). Kiwifruit ACTIN (Achn107181) and Protein Phosphatase 2A (Achn381211) were used as the reference genes for expression normalisation. The 2^{-ΔΔCt} method (Livak & Schmittgen, 2001) was used to calculate the relative expression of each gene. Primers used for qRT-PCR are provided in Table 1 below. All qPCR analyses were performed with three technical replicates.

### AsA Measurement

Measurement of total AsA concentration was performed using High-performance liquid chromatography (HPLC) following a previously described method (Queval & Noctor, 2007 Anal Biochem 363 (1): 58-69; Li et al., 2016 Plant Molecular Biology 92 (4): 473-482).

### Phylogenetic Analysis and Tree Construction

A phylogenetic tree was constructed using MEGA7.0 (Kumar et al., 2016 Mol Biol Evol 33 (7): 1870-1874) software with kiwifruit and other species sequences retrieved from kiwifruit (http://kiwifruitgenome.org/) and GenBank databases (Table 2). Genetic distances were calculated using the Jukes-Cantor distance matrix and evolutionary relationships were inferred using the neighbour-joining method with 1000 bootstrap resampling.

### Vector Construction and Kiwifruit Transformation

The coding DNA sequence (CDS) of AceGGP3, AceMYBS1, AcrMYBS1 and AceGBF3 were amplified from cDNA from A. eriantha or A. rufa and cloned into the overexpression vector (POE-3Flag-DN, from the Wuhan Botanical Garden laboratory, 35S promoter driven expression, and G418 (Geneticin, Invitrogen) or kanamycin selectable markers) to generate 35S::AceGGP3, 35S::AceMYBS1, 35S::AcrMYBS1, 35S::AceGBF3 respectively.

The AceGGP3 and AceMYBS1 were edited by CRISPR/Cas9 as described previously (Wang et al., 2018. Plant biotechnology journal 16 (8): 1424-1433). CRISPR RGEN Tools (http://www.rgenome.net/?tdsourcetag=s_pcqq_aiomsg) was used to select specific sgRNAs that targeted AceGGP3 and AceMYBS1 respectively and the sgRNAs were cloned into CRISPR/Cas9 vector to generate Cas9-AceGGP3 and Cas9-AceMYBS1 editing vectors. Using Agrobacterium-mediated transformation, the recombinant plasmids were transformed into the calli of A. eriantha following previously described methods (Akbaş et al., 2009. Appl Biochem Biotechnol 158 (2): 470-475; Yuan, 2011 Proceedings of the 2011 International Conference on Future Computer Science and Application (FCSA 2011). 17-20; Wang et al., 2018. Plant biotechnology journal 16 (4): 844-855; Wang et al., 2018. Plant biotechnology journal 16 (8): 1424-1433). Primers used for vector construction and identification of transgenic lines are listed in Table 1.

### Transient Expression Assay in Kiwifruit and Tobacco

Antisense viral vectors AceGGP3-TRV2, AceMYBS1-TRV2 and AceGBF3-TRV2 were obtained by cloning the CDS of AceGGP3, AceMYBS1, and AceGBF3 into the TRV2 vector (Yu et al., 2019a) respectively. TRV1 vector was used as an auxiliary plasmid. The TRV1 and TRV2 vectors were transformed into Agrobacterium tumefaciens GV3101A respectively and mixed with each other before injection into the calli and fruits of A. eriantha. The same method was used for transient over expression experiments with 35S driven AceGGP3, AceMYBS1 and AceGBF3 vectors in A. eriantha and Nicotiana benthamiana (tobacco) leaves.

### Yeast One-Hybrid (Y1H) Assay

Yeast one-hybrid assay was performed as previously described (Lin et al., 2007. Science 318 (5854): 1302-1305; Cheng et al., 2021. The Plant cell 33 (4): 1229-1251.). The promotor sequences of AceGGP3 (2.6-kb, Table S2) and AcrGGP3 (2.5-kb, Table S3) were amplified and inserted into the corresponding sites of the reporter plasmid pLacZi (Clontech) to generate AceGGP3 pro::LacZ and AcrGGP3pro::LacZ. The CDS of AceMYBS1 and AcrMYBS1 were separately cloned into pJG4-5 vectors (Clontech) to construct pJG-AceMYBS1 and pJG-AcrMYBS1. AceGGP3 pro::LacZ or AcrGGP3pro::LacZ were co-transformed with pJG-AceMYBS1 or pJG-AcrMYBS1 into yeast strain EGY48 using a high-efficiency yeast transformation method (Gietz & Schiestl, 2007), respectively. The p53::LacZ+pJG-p53 was used as positive control and the AceGGP3pro::LacZ or AcrGGP3pro::LacZ add the pJG4-5 empty vector were the negative control. Transformants were grew on SD/-Trp-Ura dropout medium: 6.7 g·L⁻¹ yeast nitrogen base, 20 g·L⁻¹ Galactose, 10 g·L⁻¹ Raffinose, 2 g·L⁻¹ dropout mix-TRP-URA and 20 g·L⁻¹ agar. After sterilization at 121° C. for 15 min, added 100 mL 10×BU salt (contain 70 g·L⁻¹ Na₂HPO₄·7H₂O and 30 g·L⁻¹ NaH₂PO₄, pH=7.0) and 0.08 mg·mL⁻¹ X-gal for colorimetric screening. All yeast strains were incubated at 30° C. for 3 d. Primers are listed in Table 1.

Yeast Two-Hybrid (Y2H) Assay

Construction of a cDNA library of kiwifruit for yeast two-hybrid experiments were carried out by GeneCreate Biological Engineering Co., Ltd (Wuhan, China) using mRNA from the leaf and fruit. The CDS of AceMYBS1 was cloned into the pGBKT7 vector (Clontech) as a bait plasmid. Y2H screening assays were performed as described in the BD Matchmaker Two-Hybrid Library Screening Kit user manual (Clontech). For Y2H assay, AceGBF3 was cloned from cDNA library and inserted into pGADT7 vector (Clontech). The combinations of recombinant plasmids were transferred into yeast strain Y2H gold and grown on SD/-Trp-Leu and SD/-Trp-Leu-His-Ade dropout medium plates supplemented with 0.02 mg·L⁻¹ X-α-gal, at 30° C. for 3 d. The primers are listed in Table 1.

### Transcriptional Activation Analysis

The full-length cDNA of AceMYBS1 and AceGBF3 were PCR amplified and fused into pGBKT7 vector to generated two constructed (BD-AceMYBS1 and BD-AceGBF3) and transformed into the yeast strain AH109. The transcriptional activation analysis was performed as described by (Geng & Liu, 2018. Journal of experimental botany 69 (10): 2677-2692).

### Dual-Luciferase (Dual-LUC) Assay

For the Dual-LUC assay, the full-length promoter of AceGGP3 (2.6 kb; SEQ ID No: 345) and AcrGGP3 (2.5 kb; SEQ ID No: 346) or their truncated fragments were cloned into the pGreen0800-Luc vector (Hellens et al., 2005) to obtain reporters: AceGGP3pro-2660::LUC (P₂₆₆₀), AceGGP3pro-1106::LUC (P₁₁₀₆), AceGGP3pro-1606::LUC (P₁₆₀₆), AceGGP3pro-2088::LUC (P₂₀₈₈), and AcrGGP3pro::LUC. The 35S: AceMYBS1, 35S: AcrMYBS1 and 35S: AceGBF3 were transformed into A. tumefaciens strain EHA105. Effector and reporters were mixed 5:1 (v/v) then co-infiltrated into 4-weeks-old tobacco leaves as previously (Gao et al., 2020. Journal of experimental botany 71(12): 3560-3574). After 2-3 days at 23° C., the promoter activities were determined by measuring Firefly Luciferase to Renilla Luciferase (LUC/REN) ratios using a Dual-luciferase Kit (TransGen, Beijing, China) with a Chemiluminescence Imaging System (Clinx, Shanghai, China).

### Protein Expression and Electrophoretic Mobility Shift Assay (EMSA)

The full-length AceMYBS1 CDS was inserted into the pET32a vector (Novagen) containing 6×His (both fusion in N-terminal and C-terminal) and expressed using E. coli strain BL21 (TransGen, Beijing, China) to produce recombinant AceMYBS1-His protein. An E. coli strain expressing 6×His was used as a negative control. E. coli was cultivated 12 hours at 16° C. and then diluted 1:100 (v/v) into fresh medium and grown on for another 2-3 hours at 37° C. When cell growth reached the logarithmic phase, IPTG was added to 0.5 mM final concentration and induced protein expression proceeded at 16° C. for 10-12 hours. The fusion protein was purified according to manufacturer instructions using Proteinlso Ni-NAT Resin (TransGen, Beijing, China). The oligonucleotide probes (Table S1) containing the AceMYBS1 binding sequences of AceGGP3, which was predicted by JASPAR²⁰²⁰ (http://jaspar.genereg.net/) were synthesized and labelled with biotin. Double strand DNA probes were obtained by annealing two complementary oligonucleotides. The fusion protein AceMYBS1-His was mixed with probes and incubated at room temperature for 20 min. DNA gel mobility shift assay was performed using the EMSA Kit (Beyotime, China) following the manufacturer protocol.

### Subcellular Localization

The AceMYBS1-YFP or AceGBF3-YFP mixed with NLS-mCherry-RFP were co-infiltrated into tobacco leaves with infiltration buffer by described previously (Gao et al., 2020. Journal of experimental botany 71(12): 3560-3574). Fluorescence was observed 48 h post-infiltration by Confocal Microscopy (Leica TCS-SP8; excitation wavelength with YFP: 510 nm and RFP: 552 nm).

### Bimolecular Fluorescence Complementation Assay (BiFC)

The full-length AceMYBS1 CDS was fused with C-terminal YFP (_{C}YFP), and AceGBF3 was fused with N-terminal YFP (_{N}YFP). The recombinant vectors or control (empty vectors) were transformed into A. tumefaciens strain EHA105 and then co-transformed into onion epidermal cells with infiltration buffer (Zhu et al., 2020. The Plant cell 32(10): 3155-3169). YFP fluorescence was detected by Confocal Microscopy (Leica TCS-SP8; excitation wavelength with YFP: 510 nm and DAPI: 488 nm) 48 h after infiltration.

### Pull Down

The full-length CDS of AceGBF3 was cloned it into pGEX-4T vector (GE Healthcare) in which it is fused to glutathione-S-transferase (GST) sequence. The recombinant vectors were introduced into BL21 cells to produce AceGBF3-GST fusion protein, and expressed AceGBF3-GST and AceMYBS1-6×His fusion protein was purified as described previously (Xu, 2020). Aliquots (20 μL) of AceGBF3-GST and AceMYBS1-His proteins were mixed with 1 mL binding buffer (50 mM Tris-HCl [pH=7.5], 100 mM NaCl, 0.25% Triton-X100-[v/v], 35 mM β-Mercaptoethanol), then 50 μL Proteiniso GST Resin or 50 μL Proteinlso Ni-NAT Resin (TransGen, Beijing, China) was added and the mixture was rotated at 4° C. for 3-4 hours. The samples were washed 4 times with binding buffer, with expressed GST or 6×His used as negative controls. 6×SDS protein loading buffer was added to samples (to 1× final) and samples were denatured by boiling for 10 min before electrophoresis. After electrophoresis the gels analysed by Western blot using anti-HIS (1:10,000 [v/v], Proteintech, 66005-1-Ig) and anti-GST (1:10,000 (v/v), Proteintech, 66002-2-lg) antibodies.

### Bimolecular Luminescence Complementation (BiLC) Assay

BiLC was performed as described (Chen et al., 2008. Plant physiology 146 (2): 368-376). The full-length of AceMYBS1 and AceGBF3 CDS were insert into pCAMBIA1300-cLUC and pCAMBIA1300-nLUC vector to create AceMYBS1-cLUC and AceGBF3-nLUC constructs, respectively. Agrobacterium cultures harbouring the different constructs were mixed at 1:1 (v/v) and co-transformed into tobacco leaves. Plants were incubated under dark for 12 h and then transferred to light conditions at 25° C. for 48 h. Immediately prior to luciferase activity observation, the transformed tobacco leaves were soaked in 0.15 mg·mL⁻¹ D-Luciferin potassium (Coolaber, China) for 2-3 min and images were captured using a Chemiluminescence Imaging System (Clinx, Shanghai, China).

### Statistical Analyses

One-way analysis of variance (ANOVA) was performed using SPSS v20 (IBM Corp., Armonk, NY, USA), and Student's t-test was performed using GraphPad 8.0 software. Significant differences were detected by t-tests. In the figures, the following notations are used: *, P<0.05; **, P<0.01; and ***, P<0.001. In figures, the different letters above the bars denote significance groupings (P<0.05) as determined by ANOVA, the data represent the mean values, and error bars represent standard deviations.

### Accession Numbers

The sequence information for this study was obtained from the Kiwifruit (http://kiwifruitgenome.org/), Nicotiana benthamiana (https://solgenomics.net/organism/Nicotiana benthamiana/genome) and National Center for Biotechnology Information (https://www.ncbi.nlm.nih.gov/) databases. Additionally, corresponding Acc gene models from A. chinensis (genotype Red5) are also referred to and represent PS1.1.69.0 version gene models (Pilkington et al., 2018, BMC Genomics 19). GenBank accession numbers are listed in Table 2.

TABLE 1
Primers used for qRT-PCR, vector construction and transformation
and Yeast-1-Hybrid and Yeast-2-Hybrid.

| | | SEQ | |
|---|---|---|---|
| Primers | Sequence (5′-3′) | ID | Objective |
| AcrGGP1-qpcrF1 | TGTGCTGATGATAGAAGTGGCA | 211 | qRT-PCR |
| AcrGGP1-qpcrR1 | GGGCATCATTCGCTACTTCATG | 212 | qRT-PCR |
| AcrGGP3-qpcrF1 | TGAAGCCATCTCTTGTGCTGAT | 213 | qRT-PCR |
| AcrGGP3-qpcrR1 | TGCTTCTTCACGAGATTGAGGA | 214 | qRT-PCR |
| AceGGP1-qpcrF1 | TGAAGCCATCTCTTGTGCTGAT | 215 | qRT-PCR |
| AceGGP1-qpcrR1 | AAGGGCATCATTCGCTACTTCA | 216 | qRT-PCR |
| AceGGP3-qpcrF1 | TGAAGCCATCTCTTGTGCTGAT | 217 | qRT-PCR |
| AceGGP3-qpcrR1 | TGCTTCTTCACGAGATTGAGGA | 218 | qRT-PCR |
| AcrGGP1-qpcrF2 | ATGATAGAAGTGGCAGCACGGCTGA | 219 | qRT-PCR |
| AcrGGP1-qpcrR2 | ATCATTCGCTACTTCATGAGATTGT | 220 | qRT-PCR |
| AcrGGP3-qpcrF2 | TGGAAGCGGCAGCACAGCTGA | 221 | qRT-PCR |
| AcrGGP3-qpcrR2 | ACAGTGGGAGCCTTGGTTAAG | 222 | qRT-PCR |
| AceGGP1-qpcrF2 | ATGATAGAAGCGGCAGCACGGCTGG | 223 | qRT-PCR |
| AceGGP1-qpcrR2 | ATCATTCGCTACTTCACGAGATTGA | 224 | qRT-PCR |
| AceGGP3-qpcrF2 | GGGAAGCGGCAGCACAGCTGA | 225 | qRT-PCR |
| AceGGP3-qpcrR2 | ACTGTGGGAGCCTTTGTTAAT | 226 | qRT-PCR |
| AcrGGP2-qpcrF | CCGACGGTTGTTTCCAATTACC | 227 | qRT-PCR |
| AcrGGP2-qpcrR | CTGAGGCAATTACGTCCACAAC | 228 | qRT-PCR |
| AceGGP2-qpcrF | CCGACGGTTGTTTCCAATTACC | 229 | qRT-PCR |
| AceGGP2-qpcrR | CTGAGGCAATTACGTCCACAAC | 230 | qRT-PCR |
| AceMYBS1-qpcrF | AATTCACTGGGCAGAGGAC | 231 | qRT-PCR |
| AceMYBS1-qpcrR | CATCCTCAACTAGTAATTGG | 232 | qRT-PCR |
| AcrMYBS1-qpcrF | ATGCCATTGCAATTCACTG | 233 | qRT-PCR |
| AcrMYBS1-qpcrR | TGCAGCTACATCCTCAACTA | 234 | qRT-PCR |
| AceGBF3-qpcrF | GGGACCACCACAGCCTATGATGCCA | 235 | qRT-PCR |
| AceGBF3-qpcrR | TGACATTGCTAGCCCATCGAAACCT | 236 | qRT-PCR |
| NbGGP3-qpcrF | TTTCGCATCGATCAGGTTCTTCA | 237 | qRT-PCR |
| NbGGP3-qpcrR | CTCAAACCTGAAAAGCACTTC | 238 | qRT-PCR |
| Nbβ-Tubulin-F | CAAGATGCTACTGCAGACGAG | 239 | qRT-PCR |
| Nbβ-Tubulin-R | CTGGAAGTTGTGGTTTTGGC | 240 | qRT-PCR |
| PMM-qpcrF | CCATGTGGAGTGTGATGCCAGTGTC | 241 | qRT-PCR |
| PMM-qpcrR | CAAAGCAGCAAGTTGGATGCCGTTC | 242 | qRT-PCR |
| GMP-qpcrF | GCTCTGGCTAGGGACAAACTGAT | 243 | qRT-PCR |
| GMP-qpcrR | TGGAATTCGATCATCTCTTTG | 244 | qRT-PCR |
| GME-qpcrF | ATGGTCAGCATGAATGAGATGGCCG | 245 | qRT-PCR |
| GME-qpcrR | GCTTCTCCTTAATCAGGGTGTTGTC | 246 | qRT-PCR |
| GGP-qpcrF | GAGAGCTTCTTGCTTGC | 247 | qRT-PCR |
| GGP-qpcrR | GCACCCAAGCTGTTGTAACC | 248 | qRT-PCR |
| GPP-qpcrF | ATCAGAGAAGGGCGAAGGAGACAAT | 249 | qRT-PCR |
| GPP-qpcrR | ACGAACCAGGTCAACGGGCGTCTTA | 250 | qRT-PCR |
| GLDH-qpcrF | CGGCATCGTTACCTACTACTCCTTC | 251 | qRT-PCR |
| GLDH-qpcrR | ATCTTCGGGCAATGGGGCGTAGCGG | 252 | qRT-PCR |
| GDH-qpcrF | TCAACTTCTTCGACACCTCTCCGTA | 253 | qRT-PCR |
| GDH-qpcrR | ACTCTCTCGGCACTGAAATCAAAGC | 254 | qRT-PCR |
| Actin-F | TGAGAGATTCCGTTGCCCAGAAGT | 255 | qRT-PCR |
| Actin-R | TTCCTTACTCATGCGGTCTGCGAT | 256 | qRT-PCR |
| PP2A-F | GCAGCACATAATTCCACAGG | 257 | qRT-PCR |
| PP2A-R | TTTCTGAGCCCATAACAGGAG | 258 | qRT-PCR |
| POE-AceGGP3-F | GGGACTCTAGAGGATCCATGTTGAAGATCAAGA | 259 | Over- |
| | GGGTTC | | expression |
| POE-AceGGP3-R | GTGGTACCCGGGGATCCTCAGTGCTGAACTAGG | 260 | Over- |
| | CATTC | | expression |
| POE-AceMYBS1-F | GGGACTCTAGAGGATCC | 261 | Over- |
| | ATGTCAGCTTCAGTGGATT | | expression |
| POE-AceMYBS1-R | GTGGTACCCGGGGATCCTTATTGGTGCATTGTTG | 262 | Over- |
| | GGGGTGCC | | expression |
| POE-AcrMYBS1-F | GGACTCTAGAGGATCCATGTCAGCTTCAGTGGAT | 263 | Over- |
| | T | | expression |
| POE-AcrMYBS1-R | TGGTACCCGGGGATCCTTGGTGCATTGTTGGGG | 264 | Over- |
| | GTGCC | | expression |
| POE-AceGBF3-F | GGGACTCTAGAGGATCC | 265 | over- |
| | ATGGGAAGTAGTGAAGACGTGA | | expression |
| POE-AceGBF3-R | GTGGTACCCGGGGATCC | 266 | Over- |
| | TCAGTTGACAGAGCCTG | | expression |
| POE-SnRK1A-F | GGACTCTAGAGGATCC | 267 | Over- |
| | ATGGATGGATCTGGTGGTCAAGGA | | expression |
| POE-SnRK1A-R | TGGTACCCGGGGATCC | 268 | Over- |
| | AAGAACCCGGAGCTGAGCAAGAAA | | expression |
| TRV2-AceGGP3/1-F | TAAGGTTACCGAATTCTCACTAAAGTTGGACAGG | 269 | Inhibit- |
| | AAG | | expression |
| TRV2-AceGGP3/1-R | GCTCGGTACCGGATCCAGCAGCTCAGAGATTTTC | 270 | Inhibit- |
| | A | | expression |
| TRV2-AceMYBS1-F | TAAGGTTACCGAATTCGGACAACATAGGGGTGG | 271 | Inhibit- |
| | TGG | | expression |
| TRV2-AceMYBS1-R | GCTCGGTACCGGATCCAGGAGCATTTCAAGAAA | 272 | Inhibit- |
| | CTT | | expression |
| TRV2-AceGBF3-F | TAAGGTTACCGAATTCCAGTTAGGTTGAGCTAAG | 273 | Inhibit- |
| | TC | | expression |
| TRV2-AceGBF3-R | GCTCGGTACCGGATCCTGCTGTCAAGGACAATG | 274 | Inhibit- |
| | TTG | | expression |
| Crispr-AceGGP3- | GGTCTCTTGCACTCCAGAAGTGTTGCATTCAGTT | 275 | Gene editing |
| T1F | TCAGAGCTATGC | | |
| Crispr-AceGGP3- | GGTCTCTAAACCATTCACCCTCTTGAAAGCATGC | 276 | Gene editing |
| T2R | ACCAGCCGGGAA | | |
| Crispr-AceMYBS1- | GGTCTCTTGCACGTCAATAGTGGCGATGCTTGTT | 277 | Gene editing |
| T1F | TCAGAGCTATGCTGGA | | |
| Crispr-AceMYBS1- | GGTCTCTAAACCAACATCCTCAACTAGTAATTGC | 278 | Gene editing |
| T2R | ACCAGCCGGGAATCGA | | |
| Idcrispr-AceGGP3- | AACCCAACTTAATTTCGGGCCTTTC | 279 | Identification |
| F | | | of gene edit |
| Idcrispr-AceGGP3- | ACAAACTATAAAACTGTAGAGAACG | 280 | Identification |
| R | | | of gene edit |
| Idcrispr- | GGTCTGGACAAGTTTGGGAA | 281 | Identification |
| AceMYBS1-F | | | of gene edit |
| Idcrispr- | CTGGCGTCCCAACAGCTGAT | 282 | Identification |
| AceMYBS1-R | | | of gene edit |
| YFP-AceMYBS1-F | TTTACAATTACGGATCCATGTCAGCTTCAGTGGA | 283 | Subcellular |
| | TT | | localization |
| YFP-AceMYBS1-R | CCCTTGCCCATGGATCCTTGGTGCATTGTTGGGG | 284 | Subcellular |
| | GTG | | localization |
| YFP-AceGBF3-F | TTTACAATTACGGATCCATGGGAAGTAGTGAAGA | 285 | Subcellular |
| | CGT | | localization |
| YFP-AceGBF3-R | CCCTTGCCCATGGATCCGTTGACAGAGCCTGGCT | 286 | Subcellular |
| | | | localization |
| pLaczi-AceGGP3-F | GGAATTCCGAAAAGCTCAATTAAAATGAATATA | 287 | Yeast one |
| | | | hybrid |
| pLaczi-AceGGP3-R | GGGGTACCCCCTCGAACTCAGAAAACGCAAAAA | 288 | Yeast one |
| | CA | | hybrid |
| pLaczi-AcrGGP3-F | GGAATTCCATACGACTCACTATAGGGCGAATTG | 289 | Yeast one |
| | | | hybrid |
| pLaczi-AcrGGP3-R | GGGGTACCCC | 290 | Yeast one |
| | CTCGAACTCAGAAAACGCAAAAACA | | hybrid |
| PJG-AceMYBS1-F | ATGCCTCTCCCGAATTCATGTCAGCTTCAGTGGA | 291 | Yeast one |
| | TT | | hybrid |
| PJG-AceMYBS1-R | GTCCAAAGCTTCTCGAGTTATTGGTGCATTGTTG | 292 | Yeast one |
| | GGGGTGCC | | hybrid |
| PJG-AcrMYBS1-F | ATGCCTCTCCCGAATTCATGTCAGCTTCAGTGGA | 293 | Yeast one |
| | TT | | hybrid |
| PJG-AcrMYBS1-R | GTCCAAAGCTTCTCGAGTTATTGGTGCATTGTTG | 294 | Yeast one |
| | GGGGTGCC | | hybrid |
| pGreen-AceGGP3- | GGTACCTTGAAAAGCTCAATTAAAATGAATATA | 295 | Dual-LUC |
| lianF | | | assay |
| pGreen-AceGGP3- | AAGCTTGTCGACACCGAGCTCGAATTCAAGCT | 296 | Dual-LUC |
| lianR | | | assay |
| pGreen-AceGGP3- | GGTACCTATCCCAAAATATTTATTCACTTAG | 297 | Dual-LUC |
| 2088F | | | assay |
| pGreen-AceGGP3- | GGTACCCGGATCAGCATTTGTTTTCTTCTTA | 298 | Dual-LUC |
| 1606F | | | assay |
| pGreen-AceGGP3- | TATAGGGCGAATTGGTCCTGTCGCTGACTCGCAT | 299 | Dual-LUC |
| 1106F | GAAATA | | assay |
| pGreen-AceGGP3- | TATAGGGCGAATTGG | 300 | Dual-LUC |
| 606F | TAGAAAGTGAGGTGTTGCGTCAAGA | | assay |
| pGreen-AceGGP3- | AGCTTGATATCGAATTGTCGACACCGAGCTCGAA | 301 | Dual-LUC |
| inR | TTCAAGCT | | assay |
| pGreen-AcrGGP3- | GGTACCCTAAACCTAACACAAATGGGAAGCA | 302 | Dual-LUC |
| lianF | | | assay |
| pGreen-AcrGGP3- | AAGCTTCTCGAACTCAGAAAACGCAAAAACA | 303 | Dual-LUC |
| lianR | | | assay |
| pGreen-AcrGGP3- | GGTACCCTAAACCTAACACAAATGGGAAGCA | 304 | Dual-LUC |
| 1919F | | | assay |
| pGreen-AcrGGP3- | GGTACCTCCAATCATCTCACGCCATCCAAGC | 305 | Dual-LUC |
| 1439F | | | assay |
| pGreen-AcrGGP3- | TATAGGGCGAATTGG | 306 | Dual-LUC |
| 1009F | ATCATAGGGTGGTTGGGTTGTTTGG | | assay |
| pGreen-AcrGGP3- | TATAGGGCGAATTGGGCCTCTCCTCACCTCACCC | 307 | Dual-LUC |
| 515F | CCAAAG | | assay |
| pGreen-AcrGGP3- | AGCTTGATATCGAATCTCGAACTCAGAAAACGCA | 308 | Dual-LUC |
| inR | AAAACA | | assay |
| pGreen-AceMYBS1- | CGGGGTACCGTGATTGGACCAAGAAACTGTTGG | 309 | Dual-LUC |
| F | CTCTTCTTATTGGAG | | assay |
| pGreen-AceMYBS1- | CCCAAGCTTATAGCTGGGGATTGGCACGTGTCC | 310 | Dual-LUC |
| R | GGATTCGATTGCAGC | | assay |
| AD-AceMYBS1-F | GGAGGCCAGTGAATTC | 311 | Yeast two |
| | ATGTCAGCTTCAGTGGATTG | | hybrid |
| AD-AceMYBS1-R | CGAGCTCGATGGATCC | 312 | Yeast two |
| | TTGGTGCATTGTTGGGGGTG | | hybrid |
| BD-AceMYBS1-F | CATGGAGGCCGAATTC | 313 | Yeast two |
| | ATGTCAGCTTCAGTGGATTG | | hybrid |
| BD-AceMYBS1-R | GCAGGTCGACGGATCC | 314 | Yeast two |
| | TTGGTGCATTGTTGGGGGTG | | hybrid |
| AD-AceGBF3-F | GGAGGCCAGTGAATTC | 315 | Yeast two |
| | ATGGGAAGTAGTGAAGACGT | | hybrid |
| AD-AceGBF3-R | CGAGCTCGATGGATCC GTTGACAGAGCCTGGCT | 316 | Yeast two |
| | | | hybrid |
| BD-AceGBF3-F | CATGGAGGCCGAATTC | 317 | Yeast two |
| | ATGGGAAGTAGTGAAGACGT | | hybrid |
| BD-AceGBF3-R | GCAGGTCGACGGATCC | 318 | Yeast two |
| | GTTGACAGAGCCTGGCT | | hybrid |
| AD-AceGBF3-NF | GGAGGCCAGTGAATTC | 319 | Yeast two |
| | ATGGGAAGTAGTGAAGACGT | | hybrid |
| AD-AceGBF3-NR | CGAGCTCGATGGATCC | 320 | Yeast two |
| | CATAGCAACTCCATCCACA | | hybrid |
| YCE-AceMYBS1-F | CGCCACTAGTGGATCCATGTCAGCTTCAGTGGAT | 321 | BiFC assay |
| | TGG | | |
| YCE-AceMYBS1-R | TCCCGGGAGCGGTACCTTGGTGCATTGTTGGGG | 322 | BiFC assay |
| | GTG | | |
| YNE-AceGBF3-F | CGCCACTAGTGGATCCATGGGAAGTAGTGAAGA | 323 | BiFC assay |
| | CGTG | | |
| YNE-AceGBF3-R | TCCCGGGAGCGGTACCTCAGTTGACAGAGCCTG | 324 | BiFC assay |
| | GCT | | |
| 1300cLUC- | GACGAGCTCGGTACC | 325 | BILC assay |
| AceMYBS1-F | ATGGGAAGTAGTGAAGACGTG | | |
| 1300cLUC- | ACGAGATCTGGTCGAC | 326 | BILC assay |
| AceMYBS1-R | GTTGACAGAGCCTGGCTTGT | | |
| 1300nLUC- | GACGAGCTCGGTACC | 327 | BILC assay |
| AceGBF3-F | ATGTCAGCTTCAGTGGATTGG | | |
| 1300nLUC- | ACGAGATCTGGTCGAC | 328 | BILC assay |
| AceGBF3-R | TTATTGGTGCATTGTTGGGGGTG | | |
| His-AceMYBS1-F | GGCTGATATCGGATCC | 329 | EMSA and |
| | ATGTCAGCTTCAGTGGATTG | | pull-down |
| His-AceMYBS1-R | GTGCGGCCGCAAGCTT | 330 | EMSA and |
| | TTGGTGCATTGTTGGGGGTG | | pull-down |
| GST-AceGBF3-F | GGTTCCGCGTGGATCCATGGGAAGTAGTGAAGA | 331 | Pull-down |
| | CG | | |
| GST-AceGBF3-R | CAGTCACGATGAATTCGTTGACAGAGCCTGGCTT | 332 | Pull-down |
| | G | | |
| Bio-AceMYBS1A-F | Bio-TAATTAAATAGATAAGAAAAGAGAAAAAGG | 333 | Probe of |
| | | | EMSA assay |
| Bio-AceMYBS1A-R | Bio-CCTTTTTCTCTTTTCTTATCTATTTAATTA | 334 | Probe of |
| | | | EMSA assay |
| Bio-AceMYBS1A- | Bio-TAATTAAATAAAAAAAAAAAGAGAAAAAGG | 335 | Mutated |
| mutF | | | probe of |
| | | | EMSA assay |
| Bio-AceMYBS1A- | Bio-CCTTTTTCTCTTTTTTTTTTTATTTAATTA | 336 | Mutated |
| mutR | | | probe of |
| | | | EMSA assay |
| Bio-AceMYBS1B-F | Bio-AAATAAGGGCAATCTTATCATTTATGTCAA | 337 | Probe of |
| | | | EMSA assay |
| Bio-AceMYBS1B-R | Bio-TTGACATAAATGATAAGATTGCCCTTATTT | 338 | Probe of |
| | | | EMSA assay |
| Bio-AceMYBS1B- | Bio-AAATAAGGGCAAAAAAAAAATTTATGTCAA | 339 | Mutated |
| mutF | | | probe of |
| | | | EMSA assay |
| Bio-AceMYBS1B- | Bio-TTGACATAAATTTTTTTTTTGCCCTTATTT | 340 | Mutated |
| mutR | | | probe of |
| | | | EMSA assay |
| AceMYBS1A-F | TAATTAAATAGATAAGAAAAGAGAAAAAGG | 341 | Competing |
| | | | probe of |
| | | | EMSA assay |
| AceMYBS1A-R | CCTTTTTCTCTTTTCTTATCTATTTAATTA | 342 | Competing |
| | | | probe of |
| | | | EMSA assay |
| AceMYBS1B-F | AAATAAGGGCAATCTTATCATTTATGTCAA | 343 | Competing |
| | | | probe of |
| | | | EMSA assay |
| AceMYBS1B-R | TTGACATAAATGATAAGATTGCCCTTATTT | 344 | Competing |
| | | | probe of |
| | | | EMSA assay |

TABLE 2
GenBank accession numbers.

| Gene name | Gene ID | Species |
|---|---|---|
| CaMYBS1 | KAG7023245.1 | Cucurbita argyrosperma subsp. |
| | | argyrosperma |
| MtMYBS1 | XP_013458057.1 | Medicago truncatula |
| BhMYBS1 | XP_038904415.1 | Benincasa hispida |
| JcMYBS1 | XP_012092968.1 | Jatropha curcas |
| ZmMYBS1-1 | XP_035817471.1 | Zea mays |
| ZmMYBS1-2 | XP_008655472.1 | Zea mays |
| PduMYBS1 | XP_034216346.1 | Prunus dulcis |
| SpMYBS1 | XP_015067500.1 | Solanum pennellii |
| SIMYBS1 | XP_004235740.1 | Solanum lycopersicum |
| ZjMYBS1 | XP_015883030.1 | Ziziphus jujuba |
| CisiMYBS1 | XP_006485754.1 | Citrus sinensis |
| PtMYBS1 | XP_006376751.1 | Populus trichocarpa |
| CcMYBS1 | XP_006440920.1 | Citrus clementina |
| McMYBS1 | XP_022134696.1 | Momordica charantia |
| PavMYBS1 | XP_021810789.1 | Prunus avium |
| HuMYBS1 | XP_021292289.1 | Herrania umbratica |
| CnMYBS1 | KAG1346991.1 | Cocos nucifera |
| GhMYBS1 | XP_016668580.2 | Gossypium hirsutum |
| RchMYBS1 | XP_024170989.1 | Rosa chinensis |
| AtsMYBS1-1 | XP_020182102.2 | Aegilops tauschii subsp. |
| | | Strangulata |
| AtsMYBS1-2 | XP_020153631.2 | Aegilops tauschii subsp. |
| | | Strangulata |
| AtsMYBS1-3 | XP_020147245.1 | Aegilops tauschii subsp. |
| | | Strangulata |
| EgrMYBS1 | XP_018721495.1 | Eucalyptus grandis |
| PdaMYBS1 | XP_008777770.2 | Phoenix dactylifera |
| TwMYBS1 | XP_038680313.1 | Tripterygium wilfordii |
| BrMYBS1-1 | XP_009107320.1 | Brassica rapa |
| BrMYBS1-2 | XP_009147920.1 | Brassica rapa |
| StMYBS1 | KAF7806327.1 | Senna tora |
| HaMYBS1 | XP_022017137.1 | Helianthus annuus |
| TtMYBS1 | KAF5200833.1 | Thalictrum thalictroides |
| PaMYBS1 | XP_034914820.1 | Populus alba |
| VriMYBS1 | XP_034672935.1 | Vitis riparia |
| CsaMYBS1-1 | XP_030492585.1 | Cannabis sativa |
| CsaMYBS1-2 | XP_004138233.1 | Cucumis sativus |
| PgMYBS1 | XP_031371370.1 | Punica granatum |
| PvMYBS1 | XP_031288186.1 | Pistacia vera |
| ItMYBS1 | XP_031105045.1 | Ipomoea triloba |
| QIMYBS1 | XP_030937185.1 | Quercus lobata |
| RaMYBS1 | XP_030521694.1 | Rhodamnia argentea |
| SoMYBS1 | XP_030439880.1 | Syzygium oleosum |
| AhMYBS1-1 | XP_025644418.1 | Arachis hypogaea |
| AhMYBS1-2 | XP_025616452.1 | Arachis hypogaea |
| EguMYBS1 | XP_010923684.1 | Elaeis guineensis |
| PalMYBS1-1 | XP_028753871.1 | Prosopis alba |
| PalMYBS1-2 | XP_028753870.1 | Prosopis alba |
| DcMYBS1 | XP_020701896.1 | Dendrobium catenatum |
| CasiMYBS1 | XP_028110714.1 | Camellia sinensis |
| GsMYBS1 | RZC07495.1 | Glycine soja |
| ApMYBS1 | XP_027331692.1 | Abrus precatorius |
| CaMYBS1 | XP_004508571.1 | Cicer arietinum |
| OsMYBS1-1 | XP_025882005.1 | Oryza sativa Japonica Group |
| OsMYBS1-2 | XP_ 015627636.1 | Oryza sativa Japonica Group |
| RcoMYBS1 | XP_002511974.1 | Ricinus communis |
| BdMYBS1-1 | XP_010236797.1 | Brachypodium distachyon |
| BdMYBS1-2 | XP_ 003565794.1 | Brachypodium distachyon |
| MnMYBS1 | XP_010105364.1 | Morus notabilis |
| EsMYBS1 | XP_006393319.1 | Eutrema salsugineum |
| QsMYBS1 | XP_023913108.1 | Quercus suber |
| LsMYBS1 | XP_023738581.1 | Lactuca sativa |
| CrMYBS1-1 | XP_006305410.1 | Capsella rubella |
| CrMYBS1-2 | XP_006298960.1 | Capsella rubella |
| SiMYBS1 | XP_004968851.1 | Setaria italica |
| VraMYBS1 | XP_014507110.1 | Vigna radiata var. radiata |
| BnMYBS1 | XP_013715778.1 | Brassica napus |
| HbMYBS1 | XP_021648857.1 | Hevea brasiliensis |
| MeMYBS1 | XP_021611780.1 | Manihot esculenta |
| SbMYBS1-1 | XP_002447620.2 | Sorghum bicolor |
| SbMYBS1-2 | XP_002455466.1 | Sorghum bicolor |
| AdMYBS1 | XP_015944219.1 | Arachis duranensis |
| AiMYBS1-1 | XP_016189009.1 | Arachis ipaensis |
| AiMYBS1-2 | XP_002891472.1 | Arabidopsis lyrata subsp. Lyrata |
| SinMYBS1 | XP_011073055.1 | Sesamum indicum |
| FcMYBS1 | BAG74460.1 | Fagus crenata |
| DeMYBS1-1 | KAF8730393.1 | Digitaria exilis |
| DeMYBS1-2 | KAF8670787.1 | Digitaria exilis |
| AsuGBF3.1 | KAG7644487.1 | Arabidopsis suecica |
| AsuGBF3.2 | KAG7644488.1 | Arabidopsis suecica |
| AthGBF3.1 | NP_001323456.1 | Arabidopsis thaliana |
| AthGBF3.2 | NP_001323457.1 | Arabidopsis thaliana |
| EcoGBF3 | ACC77654.1 | Eleusine coracana |
| VviGBF3 | RVX04237.1 | Vitis vinifera |
| AlyGBF3 | XP_002880229.1 | Arabidopsis lyrata subsp. lyrata |
| CroGBF3 | AAK14790.1 | Catharanthus roseus |
| AthGBF3.3 | CAA45358.1 | Arabidopsis thaliana |
| AthGBF3.4 | NP_174494.2 | Arabidopsis thaliana |
| AthGBF3.5 | AAA90947.1 | Arabidopsis thaliana |
| AthGBF3.6 | NP_850248.2 | Arabidopsis thaliana |
| AthGBF3.7 | NP_171893.1 | Arabidopsis thaliana |
| BraGBF3 | KAG5395572.1 | Brassica rapa subsp. trilocularis |
| DexGBF3.1 | KAF8695903.1 | Digitaria exilis |
| DexGBF3.2 | KAF8675825.1 | Digitaria exilis |
| DexGBF3.3 | KAF8641874.1 | Digitaria exilis |
| CsaGBF3.1 | KAF4389315.1 | Cannabis sativa |
| CsaGBF3.2 | KAF4354785.1 | Cannabis sativa |
| AthGBF3.8 | NP_171893.1 | Arabidopsis thaliana |
| TurGBF3 | EMS64924.1 | Triticum urartu |
| GarGBF3 | XP_ 017635172.1 | Gossypium arboreum |
| MnoGBF3 | EXC02957.1 | Morus notabilis |
| AceGGP3 | DTZ79_29g10040/Actinidia32270 | Actinidia eriantha/A. chinensis |
| AeGGP1 | DTZ79_17g07300/Actinidia05074 | Actinidia eriantha/A. chinensis |
| AeGGP2 | DTZ79_27g04660/Actinidia36370 | Actinidia eriantha/A. chinensis |
| AceMYBS1 | DTZ79_16g09490/Actinidia31027 | Actinidia eriantha/A. chinensis |
| AceGBF3 | DTZ79_15g00300/Actinidia27344 | Actinidia eriantha/A. chinensis |
| PMM | DTZ79_08g09640 | Actinidia eriantha |
| GMP | DTZ79_03g03560 | Actinidia eriantha |
| GME | DTZ79_24g13050 | Actinidia eriantha |

### 2. GGP3 Regulates AsA Biosynthesis in Kiwifruit

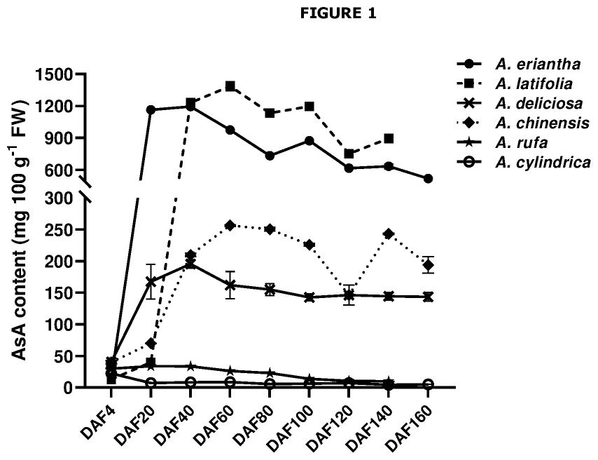

To determine the AsA variation among members of the Actinidia genus, the AsA content in the fruits of 48 species was determined by HPLC. The concentration varied tremendously among the different species, ranging from 4.4 to 1185 mg, 100 g⁻¹ FW. Species with low (0-30 mg 100.g⁻¹ FW), moderate (30-200 mg 100.g⁻¹ FW) and high vitamin C (200-1200 mg·100 g⁻¹ FW) contents constituted 29.1%, 56.3% and 14.6% of the species, respectively. Changes in AsA contents during the growing season were determined in six Actinidia species representing low, moderate and high AsA concentrations (FIG. 1). AsA content in Actinidia species with high or moderate fruit AsA content accumulated AsA rapidly after fertilization, peaking at 60 days after flowering (DAF60) sampling point, before decreasing as the fruit progressed towards maturity, whereas that of low-AsA content species stayed low level during the whole growing season. AsA biosynthesis during early fruit development is thus the main reason for AsA accumulation among members of the Actinidia genus, and differences in AsA synthesis at this stage might constitute the main determinant of AsA variation among Actinidia species.

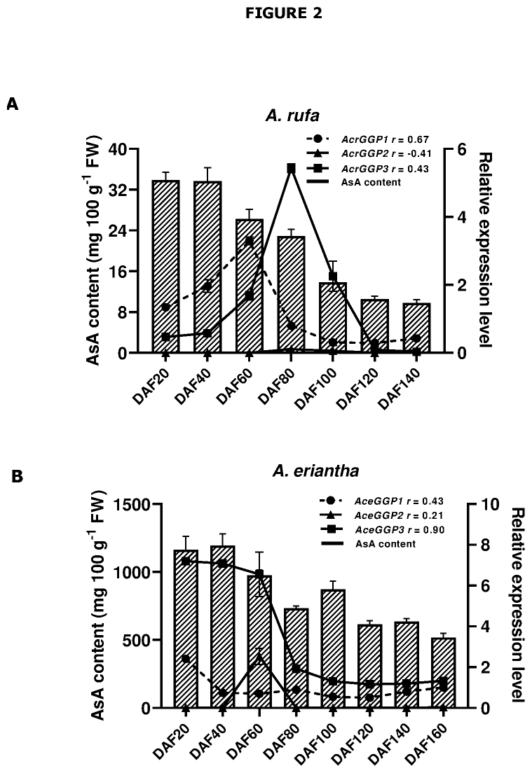

To identify potential AsA regulatory genes, transcriptome sequencing of the fruits at DAF20, DAF40, DAF60 and DAF120 was performed for A. eriantha and A. rufa, whose fruits presented a more than 30-fold difference in AsA content. A total of 24415 differentially expressed genes (DEGs) were identified by pairwise comparisons, and Gene Ontology (GO) enrichment analysis revealed that these DEGs were significantly enriched in biological processes related to the response to catalytic activity and biosynthetic processes (data not shown). The transcript Actinidia32270, encoded by GGP, was identified in A. eriantha on the basis of the increased transcript abundance in the L-galactose pathway. Three GGP homologous genes were further subjected to RT-qPCR and correlation analysis of AsA contents in the fruits of A. eriantha, A. rufa and their hybrids at different developmental stages was conducted (FIGS. 2, A and B). GGP3 in A. eriantha (AceGGP3) was most highly expressed and correlated with high AsA content in the fruits.

GGP3 expression was also highly correlated with AsA concentration in the A. eriantha x A.rufa hybrid (data not shown), and the expression of AceGGP3 allele derived from A. eriantha was significantly higher than the A. rufa derived allele AcrGGP3 (data not shown).

### 3. AceGGP3 Overexpression Leads to a Sharp Accumulation of AsA in Kiwifruit

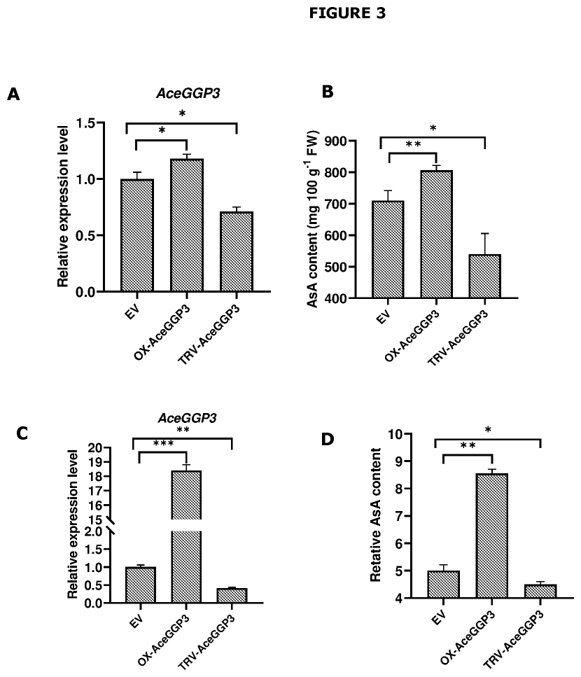

Transient expression assays were performed to confirm the function of AceGGP3 in AsA accumulation using 35S driven AceGGP3 overexpression and silencing constructs in on-vine kiwifruit and calli in tissue culture. At 7 days after infiltration AceGGP3 expression and AsA content was significantly higher infiltrated fruit than in the control fruit (FIGS. 3A and B). Moreover, silencing of AceGGP3 in fruiting plants led to an approximately 24.0% decrease in AsA compared with that in fruits infiltrated with bacteria harbouring empty vectors (FIG. 3B). Similar results were obtained in transient assays of A. eriantha calli, in which the overexpression of AceGGP3 significantly increased the AsA content (FIGS. 3C and D).

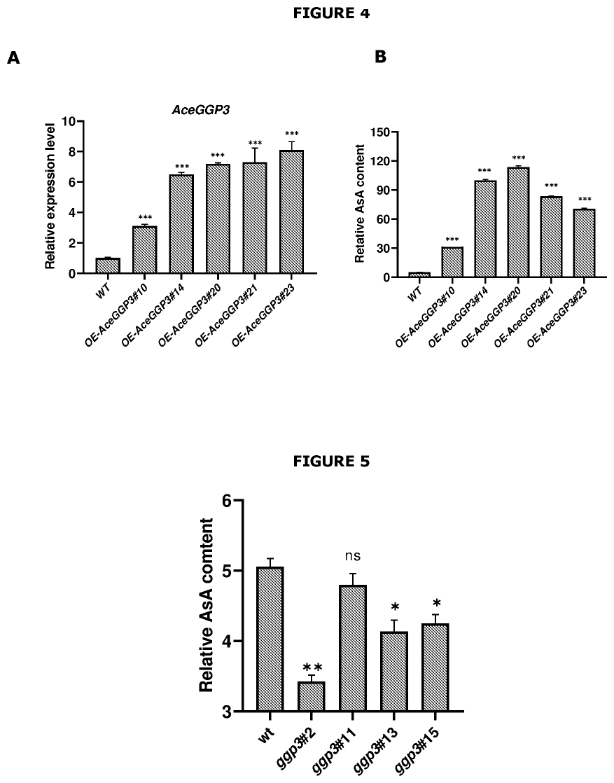

To further analyse AceGGP3 function, transgenic kiwifruit lines were generated by Agrobacterium transformation of calli. Five independent transgenic lines of A. eriantha overexpressing AceGGP3 were obtained. Compared to wild-type (WT) plants, the transgenic plants exhibited varying levels of AceGGP3 gene expression—from 3.1- to 8.1-fold (FIG. 4A), and AsA contents increased by 6.3-, 20.0-, 22.7-, 16.7- and 14.1-fold, respectively (FIG. 4B).

AceGGP3 was then mutated in kiwifruit via the CRISPR/Cas9 system, and the two targeted sites were located on the first and second exons of AceGGP3. In total, four G418-resistant lines containing three types of homozygous mutant AceGGP3 genes (ggp3 #2, a 9 bp deletion; ggp3 #11 and #15, a 1 bp insertions; ggp3 #13, a 2 bp insertion) were selected. Three mutations resulted in frameshift mutations or amino acid deletions that induced premature termination or truncation of the predicted protein, causing a loss of the AceGGP3 gene function. The AsA content in the fruits of the ggp3 mutants decreased by 32.2%, 5.11% (not statistically significant), 18.2% and 15.9% compared with that of the WT (FIG. 5). The 1 bp insertion in gg3 #11 was predicted to cause a nonsense frameshift but AsA levels remains effectively unchanged. However, a scan of ORFs in this mutated CDS showed that translation initiation from the 4th ATG codon downstream would produce a truncated version of GGP missing 53 amino acids from the N-terminus, and the unchanged AsA content in gg3 #11 suggests this variant has functional GGP activity. Collectively, the results show that up- or downregulated expression of AceGGP3 significantly affects AsA accumulation in A. eriantha.

### 4. AceMYBS1 Acts as a Transcriptional Activator of AceGGP3

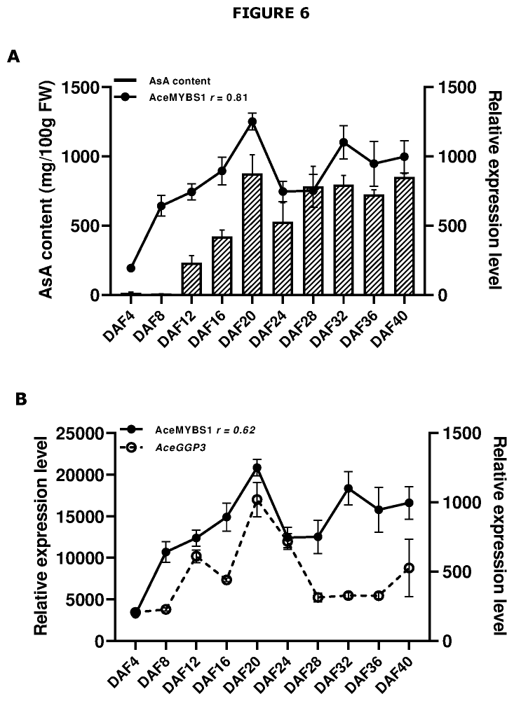

Through transcriptome analysis, a transcript, Actinidia31027 (Actinidia chinensis (Hong yang) protein v3; Actinidia39811: Actinidia chinensis (Hong yang) protein v3; Acc18653.1: LG16-18681898 . . . 18684251 [e=0]), whose expression is strongly correlated with Actinidia32270 (GGP3; SEQ ID No: 347) expression, was identified (SEQ ID No: 204), and RT-qPCR analysis also showed its expression was highly correlated with both AceGGP expression and AsA content in A. eriantha (FIGS. 6A and B). A phylogenetic tree comprising the sequences of amino acids of Actinidia31027 and MYBs of other plant species showed that Actinidia31027 was most closely related to TwMYBS1, as they shared 86% sequence identity, whereas it was most distant from CcMYB5 (not shown). Therefore, this protein has been designated as AceMYBS1. Multiple alignments performed on the SMART website (http://smart.embl-heidelberg.de/) (Schultz et al., 1998, Proceedings of the National Academy of Sciences of the United States of America 95 (11): 5857-5864; Letunic et al., 2020, Nucleic Acids Research 49 (D1): D458-D460) showed that Actinidia31027 contains two SANT/MYB regions. A SANT domain is a protein domain that allows many chromatin remodelling proteins to interact with histones (Boyer et al., 2002, Molecular Cell 10 (4): 935-942; Boyer et al., 2004, Nature Reviews Molecular Cell Biology 5 (2): 158-163), and because SANT domains share many similarities with MYB DNA binding domains and they are often conflated together. SANT and MYB domains can be distinguished by the predicted isolectric point (pI) of the domain peptide, with histone interacting SANT domains having acidic pIs, and MYB DNA binding domains having basic pI (Ko et al., 2008, Molecular Cancer 7 (1): 77). The N-terminal SANT of Actinidia31027 has a predicted acidic isoelectric point (pI), and the second SANT/MYB region has a predicted basic pI (data not shown). Therefore this predicts that AceMYBS1 has one histone interacting domain at its N-terminus side and a MYB DNA binding domain in the middle of the protein.

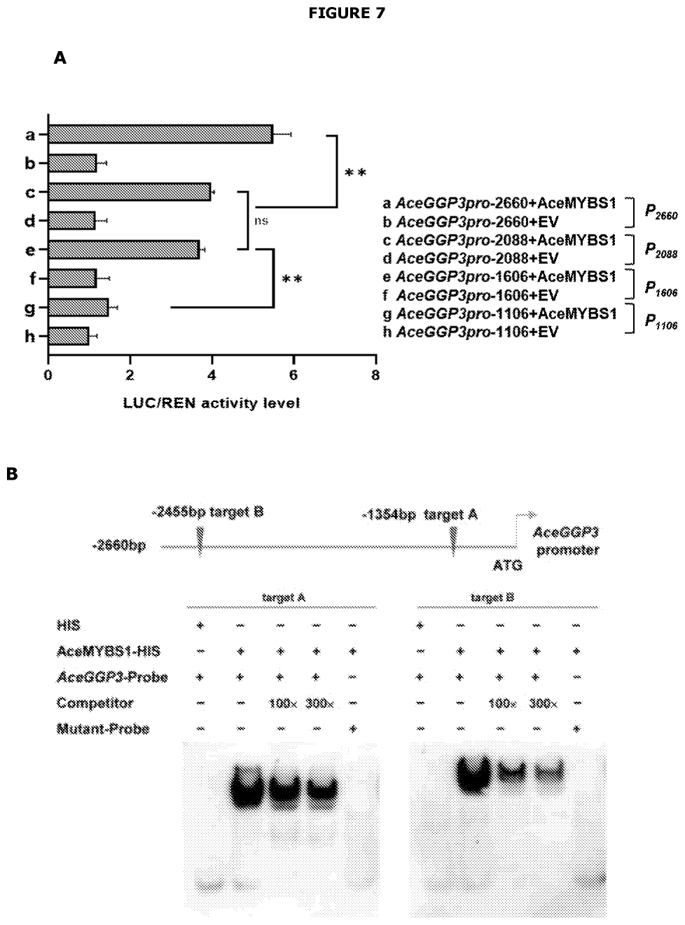

To reveal the mechanism by which AceGGP3 is regulated by AceMYBS1, a Y1H assay was conducted. All the yeast cells grew well on SD/-Trp/-Ura media, whereas only the positive control and bait vector AceGGP3pro::LacZ co-transformed with the prey vector pJG-AceMYBS1 or pJG-AcrMYBS1 had blue cells on media supplemented with X-gal (data not shown). Bioinformatics predictions were performed with JASPAR 2020 (Fornes et al., 2019, Nucleic Acids Research 48 (D1): D87-D92). Among many predicted MYBS1 cis-elements, two main cis-elements were identified within the AceGGP3 promoter (−2455 bp, TCTTATC; −1354 bp, TCTTATC). Four 5′-truncated AceGGP3 promoters (designated P₁₁₀₆, P₁₆₀₆, P₂₀₈₈, and P₂₆₆₀) were amplified from A. eriantha, and the transcriptional activity was assessed with a dual-LUC reporter system (FIG. 7). The activities of P₁₆₀₆ and P₂₆₆₀ were higher than those of P₁₁₀₆ and P₂₀₈₈, indicating that cis-elements are likely located between positions −1106-1606 bp and −2088-2660 bp, which is the same interval as the predicted position. However, regardless of which 5′-truncated AceGGP3 promoter was used to drive the reporter gene, AceMYBS1 co-transfected tobacco presented a much higher relative luciferase LUC than did AceGGP3 co-transfected tobacco, suggesting that AceMYBS1 also binds to other regions of the AceGGP3 promoter (FIG. 7A). To verify the binding specificity of AceMYBS1 to these motifs, we carried out EMSAs and found that AceMYBS1-HIS fusion proteins could bind DNA probes containing the motifs, whereas non-labelled competing probes effectively reduced the binding ability of AceMYBS1 in a dose-dependent manner, and mutation of the core sequence abolished the binding (FIG. 7B). The experiments above were repeated in A. rufa (has low AsA) and it was found that AcrMYBS1 could not bind to the AcrGGP3 promoter to regulate its expression (data not shown). The A. rufa GGP3 promoter (SEQ ID No: 346) has a number of polymorphisms (deletions/insertions) compared to that of A. eriantha (SEQ ID No: 345), including putative MYBS1 binding sites. Without wishing to be bound by theory, the investigator's hypothesise that as the AcrMYBS1 protein is almost identical to AceMYBS1 (97.7% identity at the amino acid level), the lack of binding is likely due to a defective AcrGGP3 promoter, rather than a non-functional MYBS1 protein. It may therefore be possible to increase AsA content in A. rufa by replacing or modifying the AcrGGP3 promoter, either alone or in combination with overexpression of MYBS1.

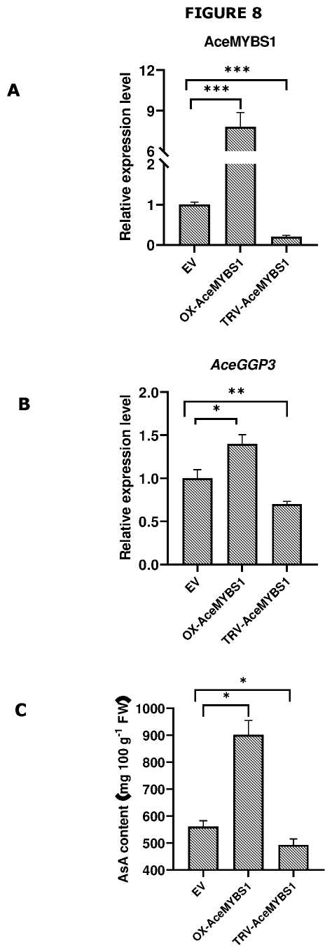

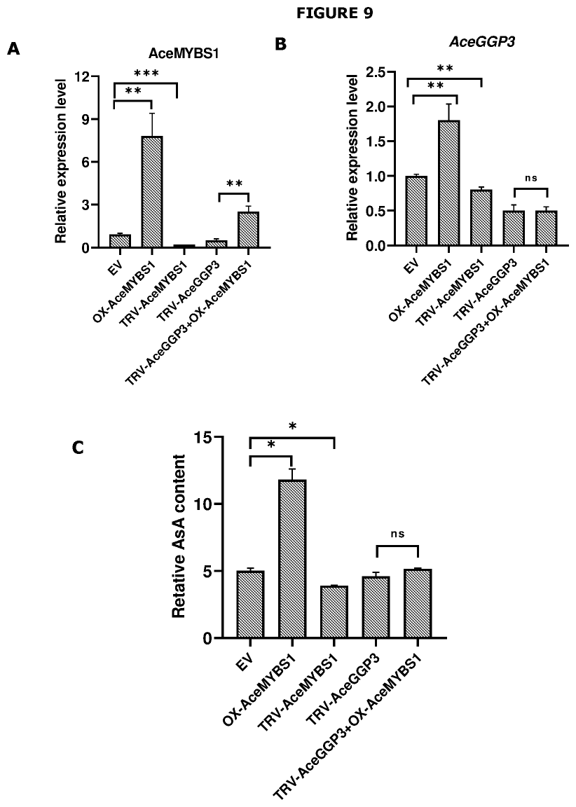

To explore the AceMYBS1 expression profiles of A. eriantha, RT-qPCR was performed to assess transcript accumulation in the fruits. Consistent with AsA content, AceMYBS1 was highly expressed in the early fruit development stage and was positively correlated with the expression of AceGGP3 (FIGS. 6A and B). A transient expression assay in A. eriantha fruits confirmed that the overexpression of AceMYBS1 increased the expression of AceGGP3 by 1.4-fold (FIGS. 8A and B), leading to a 1.7-fold increase in the AsA content in the fruits (FIG. 8C). In contrast, the expression level of AceGGP3 in the TRV-AceMYBS1 fruits decreased by 30%. As expected, overexpression of AceMYBS1 in A. eriantha calli equally led to increased expression of AceGGP3 (1.8-fold) and content of AsA (2.36-fold), and compared with empty vector-infected calli, TRV-AceMYBS1-infected calli exhibited significantly decreased AceGGP3 expression and AsA content (FIGS. 9A-C). When AceGGP3 was suppressed by TRV-AceGGP3, AsA content did not increase, even when AceMYBS1 was overexpressed in A. eriantha calli and transgenic lines, suggesting that AceMYBS1 is an upstream regulatory gene of AceGGP3.

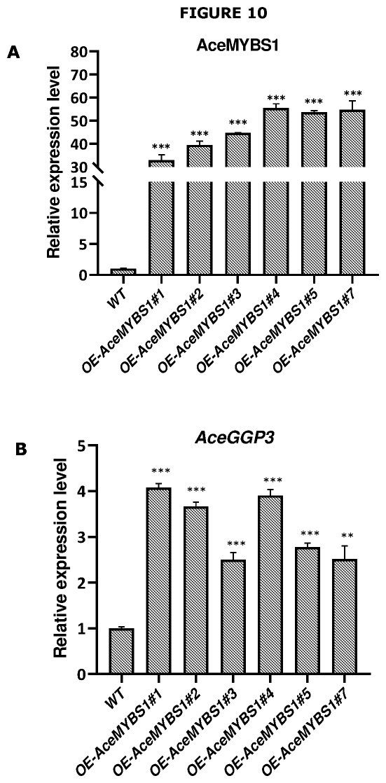

To gain further understanding of the regulatory roles of AceMYBS1, six stable overexpression transgenic kiwifruit lines were generated. RT-qPCR confirmed that the six independent transgenic lines accumulated high levels of AceMYBS1 transcripts (>32- to 55-fold; FIG. 10A), which successfully increased the expression levels of AceGGP3 by 2.5- to 4-fold (FIG. 10B). AsA content increased by 1.4-, 1.6-, 2.0-, 1.5-, 1.7- and 1.8-fold that of controls, respectively (data not shown). Using the CRISPR/Cas9 system, AceMYBS1 was further mutated in A. eriantha. The two targeted sites were located within the first and third exons. In total, five independent AceMYBS1 mutants (mybs1 #12, a 1 bp deletion; mybs1 #28, a 26 bp deletion; mybs1 #30, a 3 bp deletion; mybs1 #41, a 1 bp insertion; mybs1 #53, a 4 bp deletion) were identified (data not shown). The AsA contents of the AceMYBS1-edited lines decreased by 33.2%, 23.1%, 9.8%, 40.4% and 40.0%, respectively (data not shown). For mybs1 #30 which only had a 9.8% reduction in AsA content; while the 3 bp deletion of ATG was not in-frame, the effect was only slight because the result was effectively synonymous due to degeneracy and the only difference to WT was the loss of an aspartic acid at position 205 (just over 20 amino acids downstream from MYB DNA binding domain). Taken together, these findings support that AceMYBS1 is a positive factor that modulates AsA synthesis.

### 5. AceGBF3 Functions Additively with AceMYBS1 to Upregulate the Expression of AceGGP3 and Increase the Synthesis of AsA in Kiwifruit

To identify AceMYBS1-interacting proteins a yeast two-hybrid (Y2H) screen was conducted. Approximately 85 yeast transformants were screened, and 8 positive clones were identified as containing the same cDNA as its full-length sequence. This sequence encodes a bZIP protein (Actinidia27344; SEQ ID No: 109), whose expression was highly correlated with AceGGP3 expression (SEQ ID No: 347); this protein was then designated as AceGBF3 based on bioinformatic analysis (data not shown). Further domain mapping analysis revealed that the N-terminal region (AceGBF3-N, amino acids 1-151) but not the C-terminal half (AceGBF3-C, amino acids 152-299) of AceGBF3 interacted with AceMYBS1 (data not shown). An additional transcriptional activity assay was then performed. Yeast cells transformed with BD-AceMYBS1 or BD-AceGBF3 but not the empty pGBKT7 vector grew normally and turned blue on SD/-T/-H/-A/+X-α-Gal media (data not shown). Subcellular localization assays were performed in tobacco leaves transformed with a nuclear marker to visualize the subcellular locations of AceMYBS1 and AceGBF3. Fluorescence of the AceMYBS1 and AceGBF3-fused YFP proteins was detected only in the nucleus and perfectly merged with nuclear markers (data not shown).

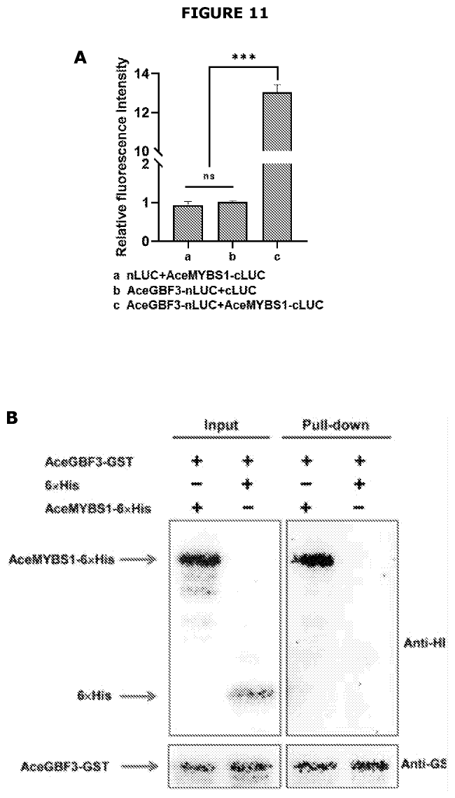

To further explore potential interactions between the AceGBF3 and AceMYBS1 proteins, three different methods were employed. Firstly, a BiFC experiment was selected as a method for an in vivo assay using a cell biology approach. AceMYBS1 and AceGBF3 proteins were fused to the C-terminus of yellow fluorescent protein (YFP) (AceMYBS1_{C}YFP) and the N-terminus of YFP (AceGBF3-_{N}YFP), respectively, and then transiently transformed into onion epidermal cells. The YFP signal localized to the nucleus showing close interaction between AceMYBS1 and AceGBF3 as well as nuclear localization (data not shown). To confirm this interaction AceMYBS1 was fused to the C-terminal half of LUC (AceMYBS1-cLUC) and AceGBF3 was fused to the N-terminal half of LUC (AceGBF3-ₙLUC) and the constructs were transiently expressed in tobacco leaves. Only leaves co-transformed with AceMYBS1-_{C}LUC and AceGBF3-ₙLUC produced a strong LUC signal (FIG. 11A). Finally, AceMYBS1-AceGBF3 physical interactions were confirmed via an in vitro pulldown assay using recombinant purified proteins. The AceGBF3-glutathione S-transferase (GST) fusion protein was precipitated with AceMYBS1-6× His but not with 6× His alone when GST resin was used (FIG. 11B).

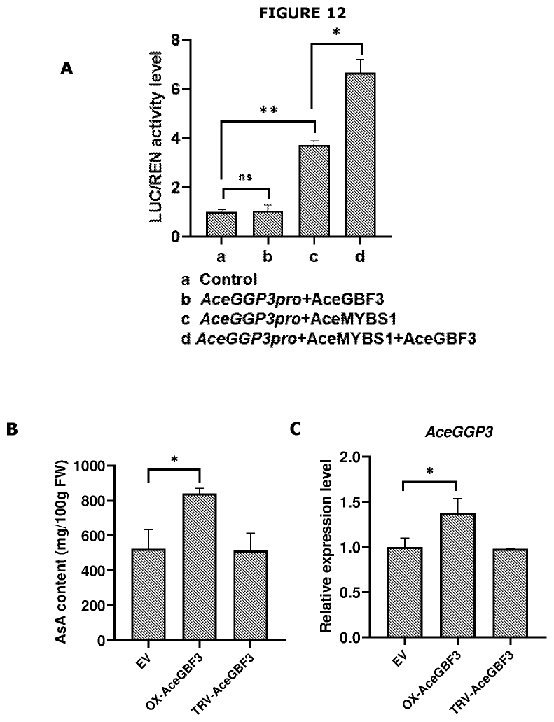

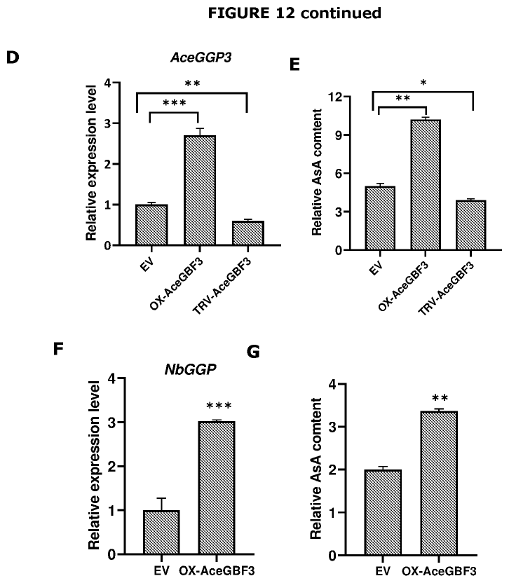

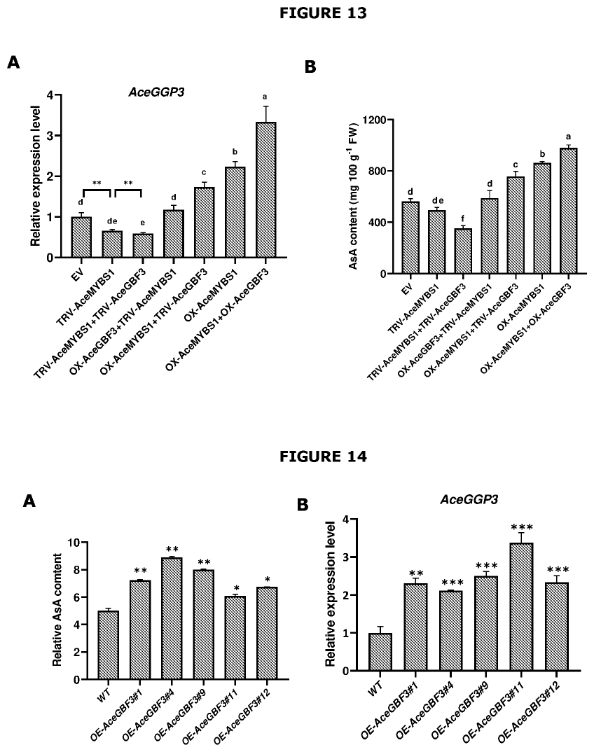

To test the hypothesis that AceGBF3 and AceMYBS1 co-regulate AsA synthesis, we performed dual-LUC, overexpression and virus-induced gene silencing experiments. These showed that AceMYBS1 and AceMYBS1 plus AceGBF3 but not AceGBF3 alone were capable of inducing the expression of AceGGP3 (of which increased by 3.74- and 6.67-fold, respectively) (FIG. 12A). Transient overexpression of AceGBF3 in different plant materials, including kiwifruit (FIG. 12B, C), calli (FIG. 12D, E) and tobacco leaves (FIG. 12, F, H), consistently promoted the accumulation of AsA and expression of GGP3. Furthermore, co-overexpression of AceGBF3 together with AceMYBS1 promoted the accumulation of AsA additively (FIG. 13A, B).

When AceMYBS1 was silenced in A. eriantha fruits, both the expression level of AceGGP3 and the content of AsA were notably reduced or unchanged, regardless of whether AceGBF3 was overexpressed or suppressed, confirming that AceGBF3 has to interact with AceMYBS1 to regulate the transcription of. Moreover, five transgenic A. eriantha lines constitutively overexpressing AceGBF3 were generated, and their AceGGP3 transcript levels and AsA contents increased by 2.11-3.38-fold and 1.22-1.78-fold, respectively, consistent with previous results (FIG. 14A, B). The average AsA content in calli (which was 2-fold higher than that in WT calli) of the AceGBF3 and AceMYBS1 co-expression lines was significantly higher than that in the calli of the AceGBF3 (1.4-fold) and AceMYBS1 (1.6-fold) lines alone (FIG. 14B). Taken together, these results show that AceGBF3 interacts with AceMYBS1 to co-regulate AceGGP3 expression to form an AceGBF3-AceMYBS1-AceGGP3 regulatory network involved in the synthesis and metabolism of AsA in kiwifruit.

### 6. AceMYBS1 Domain Structure and Genetics

The AceMYBS1 identified herein is located on chr16 and its A. chinensis and A. eriantha isoforms share 98% amino sequence identity and show no obvious perturbation in the two predicted SANT domains (data not shown). In addition to AceMYBS1 there is another highly similar MYBS1-like gene sharing 87% amino acid identity on chr26, which lies inside the recently identified A. eriantha AsA supergene QTL interval (McCallum et al., 2019, Plants (Basel, Switzerland) 8(7): 237). Alignment of A. chinensis chromosome 26 and AceMYBS1 (chr16) protein sequences revealed the chromosome 16 and 26 alleles shared highly similar SANT/MYB domains and mainly differed by a 12 amino acid deletion in the chr26 allele in an unstructured region in the C-terminal third of the protein (data not shown). Unstructured regions are typically associated with protein-protein binding interactions (Kragelund et al., 2012, Trends in Plant Science 17 (11): 625-632; Millard et al., 2019, Nucleic Acids Research 47 (18): 9592-9608). Protein disorder predictions using ODINPred (Dass et al., 2020, Scientific Reports 10 (1): 14780) also support the SMART domain architecture predictions (data not shown). The high degree of similarity, particularly between both the SANT and MYB domains suggests that the chr26 AceMYBS1 gene should also regulate GGP3 expression but it is very lowly expressed in fruit and had little correlation with AsA levels (data not shown).

### 7. Stable Expression of AceMYBS1 in Plants

### Materials and Methods

A plasmid T-DNA construct as described in Li et al. (2022, New Phytologist 235(6): 2497-2497) containing the AceMYBS1 gene (derived from Actinidia eriantha) was transformed using the Agrobacterium tumefaciens-mediated method into rice (Oryza sativa L.), soybean (Glycine max), and Arabidopsis (Arabidopsis thaliana). Stably transformed lines were selected by PCR methods. The mature leaf L-ascorbic acid content (AsA) of each of the lines (triplicate plants) was then measured using HPLC as previously described (Li et al., 2022).

### Rice (Oryza sativa L.)

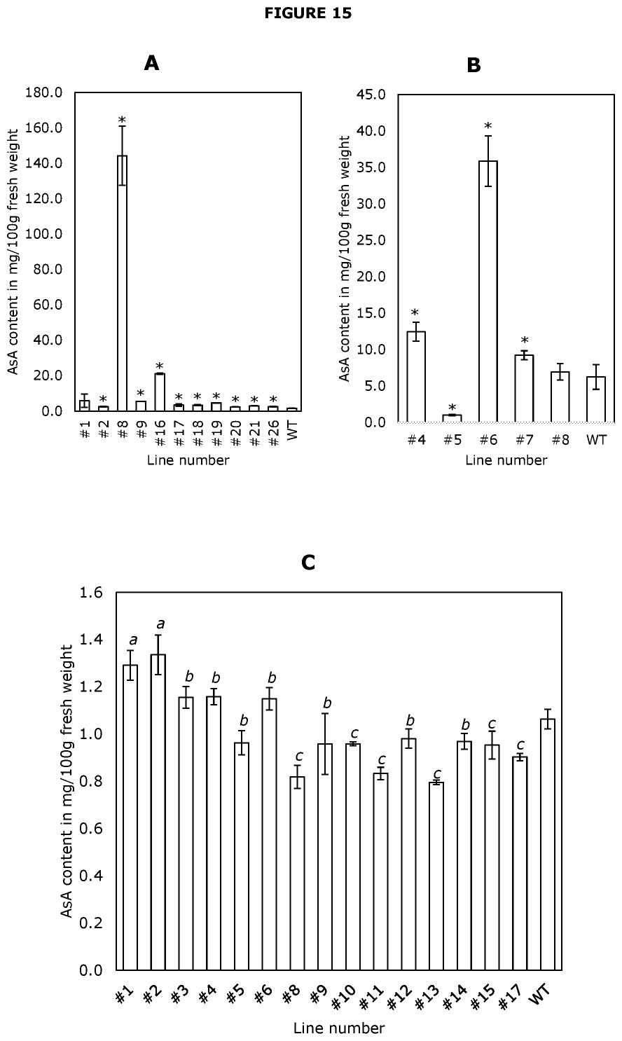

AsA content was measured in 11 transformed lines of Oryza sativa L. and compared to wild-type non-transformed controls (WT). AsA content was higher in 10 out of 11 lines (p<0.05), with variation ranging 1.5 to 89-fold that of untransformed WT (Table 3; FIG. 15A).

TABLE 3
Leaf AsA content of stably-transformed rice plants.

| Line | Mean AsA (mg/100 g | AsA content | Std | T-test |
|---|---|---|---|---|
| Number | fresh weight) | relative to WT | deviation | stat |
| #1 | 5.84 | 3.63 | 3.783 | 0.195 |
| #2 | 2.50* | 1.55 | 0.214 | 0.026 |
| #8 | 144.26* | 89.67 | 16.778 | 0.005 |
| #9 | 5.54* | 3.45 | 0.008 | 0.000 |
| #16 | 21.12* | 13.12 | 0.387 | 0.000 |
| #17 | 3.45* | 2.14 | 0.653 | 0.036 |
| #18 | 3.35* | 2.08 | 0.249 | 0.009 |
| #19 | 4.62* | 2.87 | 0.050 | 0.000 |
| #20 | 2.42* | 1.50 | 0.158 | 0.019 |
| #21 | 2.93* | 1.82 | 0.106 | 0.001 |
| #26 | 2.48* | 1.54 | 0.224 | 0.015 |
| WT | 1.61 | 1.00 | 0.040 | — |

*p < 0.05.

Soybean (Glycine max)

AsA content was measured in 5 transformed lines of Glycine max and compared to wild-type non-transformed controls (WT). AsA content was higher in 3 out of 5 lines (p<0.05), with one line having reduced AsA. Variation ranged from 0.17 to 5.7-fold that of untransformed WT (Table 4; FIG. 15B).

TABLE 4
Leaf AsA content of stably-transformed soybean plants.

| Line | Mean AsA (mg/100 g | AsA content | Std | T-test |
|---|---|---|---|---|
| Number | fresh weight) | relative to WT | deviation | stat |
| #4 | 12.46* | 1.99 | 1.287 | 0.005 |
| #5 | 1.05* | 0.17 | 0.087 | 0.035 |
| #6 | 35.88* | 5.74 | 3.464 | 0.002 |
| #7 | 9.23* | 1.48 | 0.618 | 0.040 |
| #8 | 6.96 | 1.11 | 1.133 | 0.702 |
| WT | 6.25 | 1.00 | 1.682 | — |

*p < 0.05.

Arabidopsis thaliana

The AsA content was measured in 15 transformed lines of Arabidopsis thaliana and compared to wild-type non-transformed controls (WT). Two lines displayed small increases (1.21-1.26-fold), while six lines had slightly reduced AsA (p<0.05). Variation ranged from 0.75 to 1.26-fold that of untransformed WT (Table 5; FIG. 15E). This shows that by generating a number of transformed lines and selecting plants that show the highest AsA content, it is possible to produce Arabidopsis thaliana plants having increased AsA content compared to wild-type.

TABLE 5
Leaf AsA content of stably-transformed
Arabidopsis thaliana plants.

| Line | Mean AsA (mg/100 g | AsA content | Std | T-test |
|---|---|---|---|---|
| Number | fresh weight) | relative to WT | deviation | stat |
| #1 | 1.29* | 1.21 | 0.063 | 0.020 |
| #2 | 1.34* | 1.26 | 0.084 | 0.037 |
| #3 | 1.16 | 1.09 | 0.046 | 0.102 |
| #4 | 1.16 | 1.09 | 0.034 | 0.149 |
| #5 | 0.96 | 0.91 | 0.051 | 0.125 |
| #6 | 1.15 | 1.08 | 0.047 | 0.176 |
| #8 | 0.82* | 0.77 | 0.048 | 0.041 |
| #9 | 0.96 | 0.90 | 0.129 | 0.217 |
| #10 | 0.96* | 0.90 | 0.009 | 0.042 |
| #11 | 0.83* | 0.78 | 0.027 | 0.003 |
| #12 | 0.98 | 0.92 | 0.041 | 0.220 |
| #13 | 0.80* | 0.75 | 0.010 | 0.009 |
| #14 | 0.97 | 0.91 | 0.033 | 0.057 |
| #15 | 0.95* | 0.90 | 0.059 | 0.011 |
| #17 | 0.90* | 0.85 | 0.015 | 0.024 |
| WT | 1.06 | 1.00 | 0.042 | — |

*p < 0.05.

### DISCUSSION

This example shows that constitutive over-expression of AceMYBS1 can greatly increase AsA concentration in plants. Very large increases were observed in some rice and soybean lines (˜89 and ˜5.7-fold of the wild-type control). This constitutes the largest increases in AsA observed by transgenic approaches to date (Bulley et al., 2012, Plant Biotechnology Journal 10 (4): 390-397; Macknight et al., 2017, Current Opinion in Biotechnology 44:153-160). Furthermore, as rice (monocots) and soybeans (dicots) are only distantly related, this example suggests that overexpression of MYBS1 genes can increase AsA concentration in a wide variety of plant species.

While the AsA content of individual lines varied considerably, this is not unexpected. The expression of transgenes in plants is greatly affected by the genomic context into which they are inserted, and this will vary between individual transformed lines. It is common practice to generate many transformants and select the lines that show the greatest effect. Accordingly, it is not necessary for every transformed line to show an equivalent increase in AsA content. Rather, a combination of transformation and selection can be used to provide plants with greatly increased AsA content, even if most transformed lines show only modest increases.

Over-expression of AceMYBS1 in Arabidopsis showed mixed results, but in two cases modest increases in AsA were observed. As previously stated, expression of transgenes can vary by genomic context, and this experiment confirms that it is possible to increase AsA content in Arabidopsis by over-expressing MYBS1 genes. Greater increases in AsA content may be possible by screening additional lines. This provides further evidence that the observed effect is broadly applicable to a variety of different plant species.

### 8. Expression of MYBS1 from Various Plant Species

MYBS1 genes from various plant species will be stably expressed in Arabidopsis thaliana, and the leaf AsA content measured.

The plant transformation vector pHex2s will be used, with the MYBS1 transgene being driven by a 35s constitutive promoter. The MYBS1 transgenes to be used are listed in Table 6.

TABLE 6
MYBS1 genes.

| Gene | Species | SEQ ID No |
|---|---|---|
| AceMYBS1 | Actinidia eriantha | 103 |
| AcrMYBS1 | Actinidia rufa | 357 |
| MYBS1-like | Actinidia chinensis | 108 |
| MYBS1-like | Actinidia eriantha | 358 |
| AtMYBS1 | Arabidopsis thaliana | 359 |
| AtMYBS2 | Arabidopsis thaliana | 360 |
| TaMYBS1 | Triticum aestivum | 361 |
| OsMYBS1 | Oryza sativa Japonica group | 362 |
| GmMYBS1 | Glycine max | 363 |
| CaMYBS1 | Capsicum annum | 364 |

Arabidopsis thaliana (Col0) will be transformed using Agrobacterium-mediated transformation using the floral dip method according to standard protocols.

Transformed lines will be identified and gene expression confirmed using qRT-PCR methods as described in Example 1. Transformed lines will also have their ascorbate content determined as described in Example 1, and will also be tested for increased expression of the MYBS1 target gene GGP (GDP-galactose phosphorylase) using qRT-PCR.

It is not the intention to limit the scope of the invention to the abovementioned examples only. As would be appreciated by a skilled person in the art, many variations are possible without departing from the scope of the invention as set out in the appended claims.

### INDUSTRIAL APPLICATION

The methods and tools described herein are useful for the production of plants having altered AsA content, including crops such as fruits and grains.

1-43. (canceled)

44. A method of producing a plant cell or plant with increased L-ascorbic acid (AsA) content, or increased GDP-L-galactose phosphorylase 3 (GGP) translation, production and/or activity, the method comprising either (1) transforming a plant cell with polynucleotide encoding, or upregulating in the plant cell or expressing in the plant, a polypeptide with an amino acid sequence of any one of SEQ ID NOs: 1-102 and 348-356, or a variant of the polypeptide having at least about 70% sequence identity to a polypeptide with the amino acid sequence of any one of SEQ ID NOs: 1-102 and 348-356; or (2) transforming a plant cell with, upregulating in the plant cell or expressing in the plant, a polynucleotide comprising a nucleotide sequence of any one of SEQ ID NOs: 103-108 or a variant thereof having at least 70% sequence identity to any one of SEQ ID NOs: 103-108.

45. The method of claim 44, wherein the method further comprises transforming, or co-transforming with the polynucleotide, a second polynucleotide encoding a polypeptide with an amino acid sequence of any one of SEQ ID NOs: 109-209, or a variant of the polypeptide having at least about 70% sequence identity to the amino acid sequence of any one of SEQ ID NOs: 109-209.

46. The method of claim 44, wherein the method comprises stably transforming the plant cell with the polynucleotide or variant thereof.

47. The method of claim 44, wherein the method further comprises transforming, or co-transforming with the polynucleotide, a second polynucleotide having a nucleotide sequence of SEQ ID NO: 210, or a variant thereof having at least about 70% sequence identity to the nucleotide sequence of SEQ ID NO: 210.

48. The method of claim 44, wherein the plant cell or plant is from a food or biofuel crop.

49. A genetic construct comprising a polynucleotide encoding a polypeptide with an amino acid sequence of any one of SEQ ID NOs: 1-4 and 102, or a variant of the polypeptide having at least about 75% sequence identity to the amino acid sequence of any one of SEQ ID NOs: 1-4 and 102; a polynucleotide encoding a polypeptide with an amino acid sequence of any one of SEQ ID NOs: 109-153, 155-157, 159, 162-164, 166-174, 176, 178, 180-183, 185, 186, 188-190, 192-200, 202-204, and 206-209, or a variant of the polypeptide having at least about 75% sequence identity to the amino acid sequence of any one of SEQ ID NOs: 109-153 155-157, 159, 162-164, 166-174, 176, 178, 180-183, 185, 186, 188-190, 192-200, 202-204, and 206-209; a polynucleotide comprising a nucleotide sequence of any one of SEQ ID NOs: 103-108, or a variant of the polynucleotide having at least about 75% sequence identity to the nucleotide sequence of any one of SEQ ID NOs: 103-108; or a polynucleotide comprising a nucleotide sequence of SEQ ID NO: 210, or a variant of the polypeptide having at least about 70% sequence identity to the nucleotide sequence of SEQ ID NO: 210.

50. The genetic construct of claim 49, wherein the construct comprises
a) a first polynucleotide encoding a polypeptide with an amino acid sequence of any one of SEQ ID NOs: 1-4 and 102, or a variant of the polypeptide having at least about 75% sequence identity to the amino acid sequence of any one of SEQ ID NOs: 1-4 and 102; or a polynucleotide comprising a nucleotide sequence of any one of SEQ ID NOs: 103-108, or a variant of the polynucleotide having at least about 75% sequence identity to the nucleotide sequence of any one of SEQ ID NOs: 103-108; and
b) a second polynucleotide encoding a polypeptide with an amino acid sequence of any one of SEQ ID NOs: 109-209, or a variant of the polypeptide having at least about 75% sequence identity to the amino acid sequence of any one of SEQ ID NOs: 109-209; and/or having a nucleotide sequence of SEQ ID NO: 210, or a variant of the polypeptide having at least about 70% sequence identity to the nucleotide sequence of SEQ ID NO: 210.

51. A host cell or plant cell genetically modified to express a polynucleotide encoding a polypeptide with an amino acid sequence of any one of SEQ ID NOs: 1-102 and 348-356, or a variant of the polypeptide having at least about 70% sequence identity to the amino acid sequence of any one of SEQ ID NOs: 1-102 and 348-356; or a polynucleotide comprising a nucleotide sequence of any one of SEQ ID NOs: 103-108, or a variant thereof having at least about 70% sequence identity to the nucleotide sequence of any one of SEQ ID NOs: 103-108.

52. The host cell or plant cell of claim 51, wherein the cell is genetically modified to express a second polynucleotide encoding a polypeptide with an amino acid sequence of any one of SEQ ID NOs: 109-209, or a variant of the polypeptide having at least about 70% sequence identity to the amino acid sequence of any one of SEQ ID NOs: 109-209, or a second polynucleotide having a nucleotide sequence of SEQ ID NO: 210, or a variant thereof having at least about 70% sequence identity to the nucleotide sequence of SEQ ID NO: 210.

53. The method of claim 44, wherein the polynucleotide encodes a polypeptide with, or encodes a variant of a polypeptide having at least 80, 85, 90, 95, 96, 97, 98, 99 or 100% sequence identity to, an amino acid sequence of:
c) any one of SEQ ID Nos: 1-102 and 348-356;
d) any one of SEQ ID Nos: 1-4, 38, 41-45, 48, 49, 51, 52, 56, 60, 63, 66, 79, 80, 85, 89, 90, 92, 96, 97, 100-102, and 348-356;
e) any one of SEQ ID Nos: 1-4, 49, 51, 66, 80, 85, 90, 92, 96, 102, and 348-356;
f) any one of SEQ ID Nos: 1-4 and 102;
g) any one of SEQ ID Nos: 1-4; or
h) SEQ ID No: 1 or 102.

54. The method of claim 44, wherein the polynucleotide has a nucleotide sequence of, or encodes a variant having at about 80, 85, 90, 95, 96, 97, 98, 99 or 100% sequence identity to, a nucleotide sequence of
i) any one of SEQ ID NOs: 103-108,
j) SEQ ID No: 103 or 105, or
k) SEQ ID No: 103.

55. The method of claim 45, wherein the polynucleotide or second polynucleotide encodes a polypeptide with, or a variant having at least about 70, 75, 80, 85, 90, 95, 96, 97, 98, 99 or 100% sequence identity to an amino acid sequence of:
l) any one of SEQ ID Nos: 109-209;
m) any one of SEQ ID Nos: 109-114, 125, 126, 142, 177, 179, 187, 191 and 205;
n) any one of SEQ ID Nos: 109-114 and 179;
o) any one of SEQ ID Nos: 109-114; or
p) SEQ ID NO: 109.

56. The host cell or plant cell of claim 51, wherein the polynucleotide encodes a polypeptide with, or encodes a variant of a polypeptide having at least 80, 85, 90, 95, 96, 97, 98, 99 or 100% sequence identity to an amino acid sequence of:
q) any one of SEQ ID Nos: 1-102 and 348-356;
r) any one of SEQ ID Nos: 1-4, 38, 41-45, 48, 49, 51, 52, 56, 60, 63, 66, 79, 80, 85, 89, 90, 92, 96, 97, 100-102, and 348-356;
s) any one of SEQ ID Nos: 1-4, 49, 51, 66, 80, 85, 90, 92, 96, 102, and 348-356;
t) any one of SEQ ID Nos: 1-4 and 102;
u) any one of SEQ ID Nos: 1-4; or
v) SEQ ID No: 1 or 102.

57. The host cell or plant cell of claim 51, wherein the polynucleotide has a nucleotide sequence of, or encodes a variant having at about 80, 85, 90, 95, 96, 97, 98, 99 or 100% sequence identity to a nucleotide sequence of
w) any one of SEQ ID NOs: 103-108,
x) SEQ ID No: 103 or 105, or
y) SEQ ID No: 103.

58. The host cell or plant cell of claim 52, wherein the polynucleotide or second polynucleotide encodes a polypeptide with, or a variant having at least about 70, 75, 80, 85, 90, 95, 96, 97, 98, 99 or 100% sequence identity to an amino acid sequence of:
z) any one of SEQ ID Nos: 109-209;
aa) any one of SEQ ID Nos: 109-114, 125, 126, 142, 177, 179, 187, 191 and 205;
bb) any one of SEQ ID Nos: 109-114 and 179;
cc) any one of SEQ ID Nos: 109-114; or
dd) SEQ ID NO: 109.
